(19) United States
(12) Patent Application Publication
(10) Pub. No.: US 2012/0071506 A1
(43) Pub. Date: Mar. 22, 2012

(54) METHODS FOR TREATING CNS DISORDERS WITH BICYCLO-SUBSTITUTED 2-IMIDAZOLINE AND 2-IMIDAZOLES

(76) Inventors: Guido Galley, Rheinfelden (DE); Katrin Groebke Zbinden, Liestal (CH); Roger Norcross, Olsberg (CH); Henri Stalder, Basel (CH)

(21) Appl. No.: 13/303,465

(22) Filed: Nov. 23, 2011

Related U.S. Application Data
(63) Continuation of application No. 11/655,375, filed on Jan. 19, 2007, now abandoned.

(30) Foreign Application Priority Data
Jan. 27, 2006 (EP) 06100951.0

(51) Int. Cl. A61K 31/4709; A61K 31/4178; C07D 401/04; C07D 401/06; C07D 405/04; C07D 233/60; C07D 233/58; C07D 233/20; A61P 25/20; C07D 233/06; C07D 233/24; C07D 405/06; C07D 233/22; A61P 25/24; A61P 25/22; A61P 25/18; A61P 25/00; A61P 25/16; A61P 25/28; A61P 25/08; A61P 25/06; A61P 9/12; A61P 25/30; A61P 3/00; A61P 3/10; A61P 3/04; A61P 3/06; A61P 43/00; A61P 9/00; A61K 31/4164

(52) U.S. Cl. 514/312; 514/314; 514/396; 514/397; 514/401; 514/402; 546/153; 546/165; 546/176; 548/311.4; 548/341.1; 548/345.1; 548/347.1

(57) ABSTRACT

The present invention relates to a method for treating a disorder selected from depression, anxiety disorders, bipolar disorder, attention deficit hyperactivity disorder, stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders which comprises administering to an individual a therapeutically effective amount of a compound of formula I

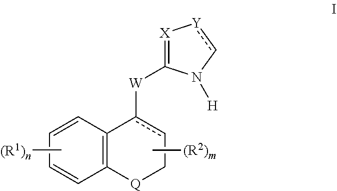

wherein
R¹, R², Q, W, X, Y, m, and n are as defined in the specification, and their pharmaceutically active salts, racemic mixtures, enantiomers, optical isomers and tautomeric forms. The invention also relates to novel compounds of formula I, compositions containing them, and methods for their preparation.

### PRIORITY TO RELATED APPLICATIONS

This application is a continuation of U.S. application Ser. No. 11/655,375, filed Jan. 19, 2007, now pending; which claims the benefit of European Application No. 06100951.0, filed Jan. 27, 2006. The entire contents of the above-identified applications are hereby incorporated by reference.

### BACKGROUND OF THE INVENTION

The classical biogenic amines (serotonin, norepinephrine, epinephrine, dopamine, histamine) play important roles as neurotransmitters in the central and peripheral nervous system [1]. Their synthesis and storage, as well as their degradation and reuptake after release are tightly regulated. An imbalance in the levels of biogenic amines is known to be responsible for the altered brain function under many pathological conditions [2-5]. A second class of endogenous amine compounds, the so-called trace amines (TAs) significantly overlap with the classical biogenic amines regarding structure, metabolism and subcellular localization. The TAs include p-tyramine, β-phenylethylamine, tryptamine and octopamine, and they are present in the mammalian nervous system at generally lower levels than classical biogenic amines [6].

Their dysregulation has been linked to various psychiatric diseases like schizophrenia and depression [7] and for other conditions like attention deficit hyperactivity disorder, migraine headache, Parkinson's disease, substance abuse and eating disorders [8,9].

For a long time, TA-specific receptors had only been hypothesized based on anatomically discrete high-affinity TA binding sites in the CNS of humans and other mammals [10,11]. Accordingly, the pharmacological effects of TAs were believed to be mediated through the well known machinery of classical biogenic amines, by either triggering their release, inhibiting their reuptake or by “crossreacting” with their receptor systems [9,12,13]. This view changed significantly with the recent identification of several members of a novel family of GPCRs, the trace amine associated receptors (TAARs) [7,14]. There are 9 TAAR genes in human (including 3 pseudogenes) and 16 genes in mouse (including 1 pseudogene). The TAAR genes do not contain introns (with one exception, TAAR2 contains 1 intron) and are located next to each other on the same chromosomal segment. The phylogenetic relationship of the receptor genes, in agreement with an in-depth GPCR pharmacophore similarity comparison and pharmacological data suggest that these receptors form three distinct subfamilies [7,14]. TAAR1 is in the first subclass of four genes (TAAR1-4) highly conserved between human and rodents. TAs activate TAAR1 via Gαs. Dysregulation of TAs was shown to contribute to the aetiology of various diseases like depression, psychosis, attention deficit hyperactivity disorder, substance abuse, Parkinson's disease, migraine headache, eating disorders, metabolic disorders and therefore TAAR1 ligands have a high potential for the treatment of these diseases.

Therefore, there is a broad interest to increase the knowledge about trace amine associated receptors.

### SUMMARY OF THE INVENTION

The present invention provides a method for treating a disorder selected from the group consisting of depression, anxiety disorders, bipolar disorder, attention deficit hyperactivity disorder, stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders which comprises administering to an individual a therapeutically effective amount of a compound of formula I

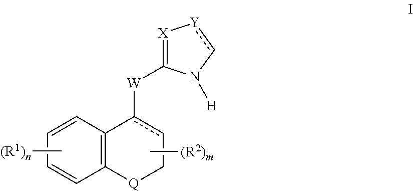

wherein
R¹ is hydrogen, tritium, hydroxy, lower alkyl, lower alkoxy, halogen, nitro, amino or lower alkyl substituted by halogen;
R² is hydrogen, hydroxy or lower alkyl;
X is N and Y is CH or CH₂ or CH-lower alkyl or
X is CH and Y is N;
Q is CH₂, O, NH, N-alkyl, N—SO₂-alkyl or N—SO₂-toluen-4-yl;
W is CH₂ or a bond
m and n are each independently 1, 2 or 3; when m is 2 or 3, each R² is the same or different; when n is 2 or 3, each R¹ is the same or different;
the dotted lines each independently represent an optional bond;
   and their pharmaceutically active salts, racemic mixtures, enantiomers, optical isomers and tautomeric forms.

The compounds of formula I have a good affinity to the trace amine associated receptors (TAARs), especially for TAAR1.

Some of the compounds of formula I are known compounds, described for example in the below mentioned references, or are disclosed in public chemical libraries.

### REFERENCES USED

1 Deutch, A. Y. and Roth, R. H. (1999) Neurotransmitters. In Fundamental Neuroscience (2^{nd} edn) (Zigmond, M. J., Bloom, F. E., Landis, S. C., Roberts, J. L, and Squire, L. R., eds.), pp. 193-234, Academic Press;
2 Wong, M. L. and Licinio, J. (2001) Research and treatment approaches to depression. Nat. Rev. Neurosci. 2, 343-351;
3 Carlsson, A. et al. (2001) Interactions between monoamines, glutamate, and GABA in schizophrenia: new evidence. Annu. Rev. Pharmacol. Toxicol. 41, 237-260;
4 Tuite, P. and Riss, J. (2003) Recent developments in the pharmacological treatment of Parkinson's disease. Expert Opin. Investig. Drugs 12, 1335-1352,
5 Castellanos, F. X. and Tannock, R. (2002) Neuroscience of attention-deficit/hyperactivity disorder: the search for endophenotypes. Nat. Rev. Neurosci. 3, 617-628;
6 Usdin, E. and Sandler, M. eds. (1984), Trace Amines and the brain, Dekker;
7 Lindemann, L. and Hoener, M. (2005) A renaissance in trace amines inspired by a novel GPCR family. Trends in Pharmacol. Sci. 26, 274-281;
8 Branchek, T. A. and Blackburn, T. P. (2003) Trace amine receptors as targets for novel therapeutics: legend, myth and fact. Curr. Opin. Pharmacol. 3, 90-97;
9 Premont, R. T. et al. (2001) Following the trace of elusive amines. Proc. Natl. Acad. Sci. U. S.A. 98, 9474-9475;
10 Mousseau, D. D. and Butterworth, R. F. (1995) A high-affinity [3H] tryptamine binding site in human brain. Prog. Brain Res. 106, 285-291;
11 McCormack, J. K. et al. (1986) Autoradiographic localization of tryptamine binding sites in the rat and dog central nervous system. J. Neurosci. 6, 94-101;
12 Dyck, L. E. (1989) Release of some endogenous trace amines from rat striatal slices in the presence and absence of a monoamine oxidase inhibitor. Life Sci. 44, 1149-1156;
13 Parker, E. M. and Cubeddu, L. X. (1988) Comparative effects of amphetamine, phenylethylamine and related drugs on dopamine efflux, dopamine uptake and mazindol binding. J. Pharmacol. Exp. Ther. 245, 199-210;
14 Lindemann, L. et al. (2005) Trace amine associated receptors form structurally and functionally distinct subfamilies of novel G protein-coupled receptors. Genomics 85, 372-385.

Compounds of examples 1-14, 26-55 and 57-74 are novel. Thus, the invention also provides for these compounds.

The invention also provides methods for the production of compounds of the invention and pharmaceutical compositions containing them.

The invention also provides methods for using a labeled compound of formula I as a radioligand in a binding assay for trace amine associated receptors.

The preferred indications the present invention are depression, psychosis, Parkinson's disease, anxiety and attention deficit hyperactivity disorder (ADHD).

The invention provides novel compounds of formula I

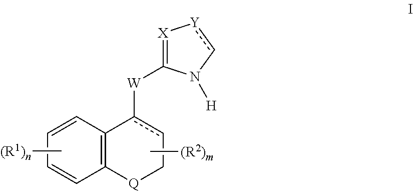

wherein
R¹ is hydrogen, tritium, hydroxy, lower alkyl, lower alkoxy, halogen, nitro, amino or lower alkyl substituted by halogen;
R² is hydrogen, hydroxy or lower alkyl;
X is N and Y is CH or CH₂ or CH-lower alkyl or
X is CH and Y is N;
Q is CH₂, O, NH, N-alkyl, N—SO₂-alkyl or N—SO₂-toluen-yl;
W is CH₂ or a bond
m and n are independently 1, 2 or 3; when m is 2 or 3, each R² is the same or different; when n is 2 or 3, each R¹ is the same or different;
the dotted lines each independently represents an optional bond;
   and their pharmaceutically active salts, racemic mixtures, enantiomers, optical isomers and tautomeric forms, with the exception of the following compounds
rac-2-(1,2,3,4-tetrahydro-1-naphthyl)-2-imidazoline
rac-2-(7-methyl-1,2,3,4-tetrahydro-naphthalen-1-yl)-4,5-dihydro-1H-imidazole
rac-2-(6-methyl-1,2,3,4-tetrahydro-naphthalen-1-yl)-4,5-dihydro-1H-imidazole
rac-2-(6-chloro-1,2,3,4-tetrahydro-naphthalen-1-yl)-4,5-dihydro-1H-imidazole
rac-2-(5-chloro-1,2,3,4-tetrahydro-naphthalen-1-yl)-4,5-dihydro-1H-imidazole
rac-2-(7-methoxy-1,2,3,4-tetrahydro-naphthalen-1-yl)-4,5-dihydro-1H-imidazole
rac-2-(6-methoxy-1,2,3,4-tetrahydro-naphthalen-1-yl)-4,5-dihydro-1H-imidazole
rac-2-(5-methoxy-1,2,3,4-tetrahydro-naphthalen-1-yl)-4,5-dihydro-1H-imidazole
rac-5-(4,5-dihydro-1H-imidazol-2-yl)-5,6,7,8-tetrahydro-naphthalen-2-ol,
rac-4-(1,2,3,4-tetrahydro-naphthalen-1-yl)-1H-imidazole
rac-5-(4,5-dihydro-1H-imidazol-2-yl)-5,6,7,8-tetrahydro-naphthalene-2,3-diol and
rac-5-(4,5-dihydro-1H-imidazol-2-yl)-5,6,7,8-tetrahydro-naphthalene-1,2-diol.

The novel compounds of formula I can also be used as radioligands in a binding assay for trace amine associated receptors.

### DETAILED DESCRIPTION OF THE INVENTION

The following definitions of general terms used in the present description apply irrespective of whether the terms in question appear alone or in combination. It must be noted that, as used in the specification and the appended claims, the singular forms “a”, “an,” and “the” include plural forms unless the context clearly dictates otherwise.

As used herein, the term “lower alkyl” denotes a saturated straight- or branched-chain hydrocarbon group containing from 1 to 7 carbon atoms, for example, methyl, ethyl, propyl, isopropyl, n-butyl, i-butyl, 2-butyl, t-butyl and the like. Preferred alkyl groups are groups with 1-4 carbon atoms.

As used herein, the term “lower alkoxy” denotes a group wherein the alkyl residue as defined above which is attached via an oxygen atom.

As used herein, the term “lower alkyl substituted by halogen” denotes an alkyl group as defined above, wherein at least one hydrogen atom is replaced by halogen, for example CF₃, CHF₂, CH₂F, CH₂CF₃, CH₂CF₂CF₃ and the like.

The term “halogen” denotes chlorine, iodine, fluorine and bromine.

“Pharmaceutically acceptable” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

In one embodiment, the invention provides a method for treating a disorder selected form the group consisting of depression, anxiety disorders, bipolar disorder, attention deficit hyperactivity disorder, stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders which comprises administering to an individual, a therapeutically effective amount of a compound of formula IA

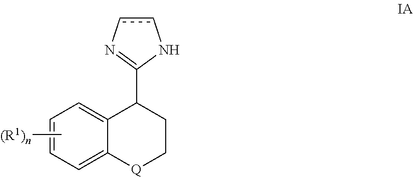

wherein
R¹ is hydrogen, tritium, hydroxy, lower alkyl, lower alkoxy, halogen or lower alkyl substituted by halogen;
Q is CH₂ or O;
n is 1, 2 or 3; when n is 2 or 3, each R¹ is the same or different;
the dotted line represents an optional bond;
   and their pharmaceutically active salts, racemic mixtures, enantiomers, optical isomers and tautomeric forms.

Within this method, preferred compounds of formula I are those, wherein X is N.

Preferred compounds from this group are those, wherein Q is CH₂ and R¹ is halogen, for example the following compounds:
rac-2-(5-bromo-1,2,3,4-tetrahydro-naphthalen-1-yl)-4,5-dihydro-1H-imidazole
rac-2-(7-chloro-5-fluoro-1,2,3,4-tetrahydro-naphthalen-1-yl)-4,5-dihydro-1H-imidazole
rac-2-(6-chloro-1,2,3,4-tetrahydro-naphthalen-1-yl)-4,5-dihydro-1H-imidazole and
rac-2-(5-chloro-1,2,3,4-tetrahydro-naphthalen-1-yl)-4,5-dihydro-1H-imidazole.

Additional preferred compounds of formula I for use in the method of the invention are those, wherein Q is CH₂ and R¹ is lower alkyl, for example the following compounds:
rac-2-(5,7-dimethyl-1,2,3,4-tetrahydro-naphthalen-1-yl)-4,5-dihydro-1H-imidazole and
rac-2-(5,7-dimethyl-1,2,3,4-tetrahydro-naphthalen-1-yl)-1H-imidazole.

Further preferred compounds of formula I for use in the method of the invention are those, wherein Q is CH₂ and R¹ is lower alkoxy, for example the following compounds:
rac-2-(7-methoxy-1,2,3,4-tetrahydro-naphthalen-1-yl)-4,5-dihydro-1H-imidazole
rac-2-(6-methoxy-1,2,3,4-tetrahydro-naphthalen-1-yl)-4,5-dihydro-1H-imidazole and
rac-2-(5-methoxy-1,2,3,4-tetrahydro-naphthalen-1-yl)-4,5-dihydro-1H-imidazole.

Other preferred compounds of formula I for use in the method of the invention also are those, wherein Q is O or NH and R¹ is hydrogen or halogen, for example
rac-2-(6,8-dichloro-chroman-4-yl)-1H-imidazole and
rac-4-(1H-imidazol-2-yl)-1,2,3,4-tetrahydro-quinoline.

Also preferred for use in the method of the invention are compounds, wherein X is CH.

Further preferred are compounds from this group are those, wherein Q is CH₂ and R¹ is hydrogen, for example the following compounds:
(4-(3,4-dihydro-naphthalen-1-yl)-1H-imidazole and
rac-4-(1,2,3,4-tetrahydro-naphthalen-1-yl)-1H-imidazole.

Preferred compounds from this group also are those, wherein Q is O and R¹ is hydrogen, for example the following compound:
rac-5-chroman-4-yl-1H-imidazole hydrochloride or tautomer.

Preferred compounds from this group are further those, wherein Q is O and R¹ is lower alkyl, for example the following compounds:
rac-5-(7-methyl-chroman-4-yl)-1H-imidazole or tautomer and
rac-5-(5-methyl-chroman-4-yl)-1H-imidazole or tautomer.

Preferred compounds from this group are further those, wherein Q is O and R¹ is halogen, for example the following compounds:
rac-5-(6-fluoro-chroman-4-yl)-1H-imidazole or tautomer
5-(8-chloro-2H-chromen-4-yl)-1H-imidazole or tautomer
5-(6-chloro-2H-chromen-4-yl)-1H-imidazole or tautomer
rac-5-(7-fluoro-chroman-4-yl)-1H-imidazole or tautomer and
rac-5-(5-fluoro-chroman-4-yl)-1H-imidazole or tautomer.

Preferred novel compounds are the following:

Compounds of formula I, wherein X is N, Q is CH₂ and R¹ is halogen, for example the following compounds
rac-2-(5-bromo-1,2,3,4-tetrahydro-naphthalen-1-yl)-4,5-dihydro-1H-imidazole and
rac-2-(7-chloro-5-fluoro-1,2,3,4-tetrahydro-naphthalen-1-yl)-4,5-dihydro-1H-imidazole.
   Compounds of formula I, wherein X is N, Q is CH₂ and R¹ is tritium, for example
rac-2-(7-tritio-1,2,3,4-tetrahydro-naphthalen-1-yl)-4,5-dihydro-1H-imidazole.
   Compounds of formula I, wherein X is N and Q is —O—, for example the following compounds
rac-2-chroman-4-yl-4,5-dihydro-1H-imidazole,
rac-2-chroman-4-yl-1H-imidazole and
rac-2-(6-fluoro-chroman-4-yl)-1H-imidazole.
   Compounds of formula I, wherein X is N, Q is O or NH and R¹ is hydrogen or halogen, for example
rac-2-(6,8-dichloro-chroman-4-yl)-1H-imidazole and
rac-4-(1H-imidazol-2-yl)-1,2,3,4-tetrahydro-quinoline.
   Compounds of formula I, wherein X is CH, Q is CH₂ and R¹ is hydrogen, for example the following compound:
(4-(3,4-dihydro-naphthalen-1-yl)-1H-imidazole.
   Compounds of formula I, wherein X is CH, Q is O and R¹ is hydrogen, for example the following compound:
rac-5-chroman-4-yl-1H-imidazole hydrochloride or tautomer.
   Compounds of formula I, wherein X is CH, Q is O and R¹ is lower alkyl, for example the following compounds:
rac-5-(7-methyl-chroman-4-yl)-1H-imidazole or tautomer and
rac-5-(5-methyl-chroman-4-yl)-1H-imidazole or tautomer.
   Compounds of formula I, wherein X is CH, Q is O and R¹ is halogen, for example the following compounds:
rac-5-(6-fluoro-chroman-4-yl)-1H-imidazole or tautomer
5-(8-chloro-2H-chromen-4-yl)-1H-imidazole or tautomer
5-(6-chloro-2H-chromen-4-yl)-1H-imidazole or tautomer
rac-5-(7-fluoro-chroman-4-yl)-1H-imidazole or tautomer and
rac-5-(5-fluoro-chroman-4-yl)-1H-imidazole or tautomer.

The present compounds of formula I and their pharmaceutically acceptable salts can be prepared by methods known in the art, for example, by processes described below, which process comprises

a) reacting a compound of formula

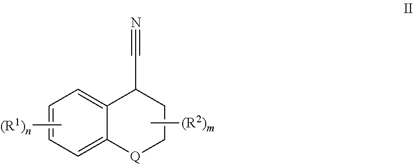

with ethylenediamine of formula

H₂NCH₂CH₂NH₂ III

to obtain a compound of formula

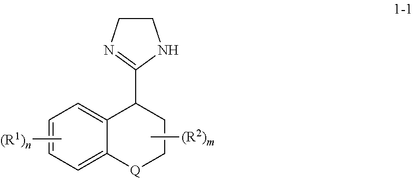

wherein R¹, R², Q, m and n are as defined above, or
b) reducing a compound of formula

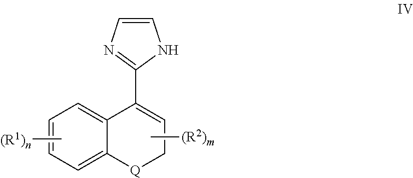

by catalytic hydrogenation in the presence of Pd/C or by a complex hydride
to obtain a compound of formula

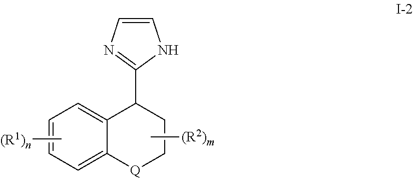

wherein R¹, R², Q, m and n are as defined above are as defined above, or
c) reducing a compound of formula

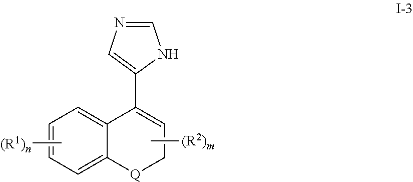

by catalytic hydrogenation in the presence of Pd/C or by a complex hydride
to obtain a compound of formula

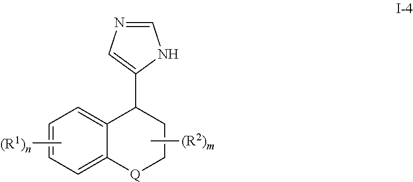

wherein R¹, R², Q, m and n are as defined above are as defined above, or
d) deprotecting a compound of formula

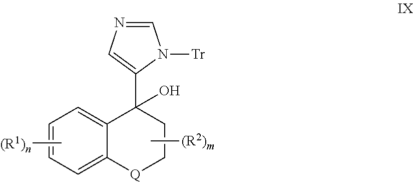

with formic acid
to obtain a compound of formula

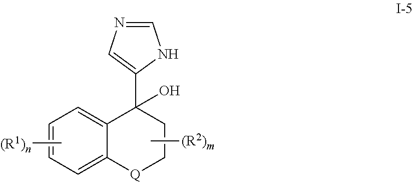

wherein R¹, R², Q, m and n are as defined above are as defined above, or
e) reacting a compound of formula

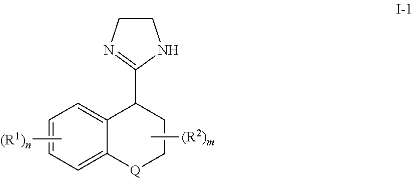

with DMSO and oxalyl chloride in dichloromethane or permanganate absorbed on silica gel in acetonitrile or with Pd/C in toluene
to obtain a compound of formula

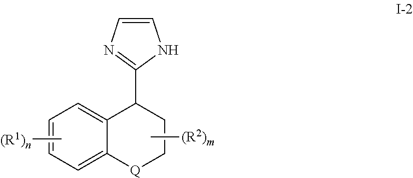

wherein R¹, R², Q, m and n are as defined above, or
f) reacting a compound of formula

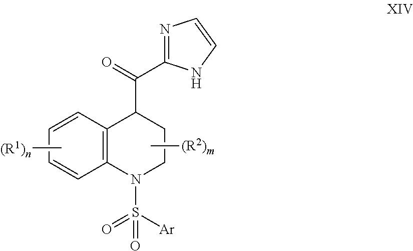

with NaOH and hydrazine hydrate
to obtain a compound of formula

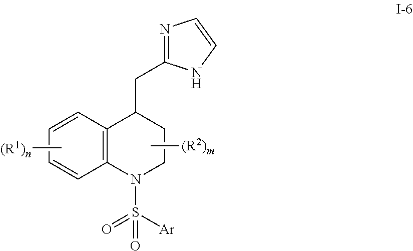

wherein R¹, R², m and n are as defined above, or
g) reacting a compound of formula

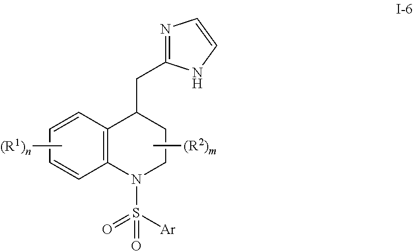

with HBr, acetic acid and anisole
to obtain a compound of formula

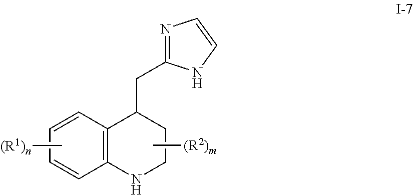

wherein R¹, R², m and n are as defined above, or
h) reacting a compound of formula

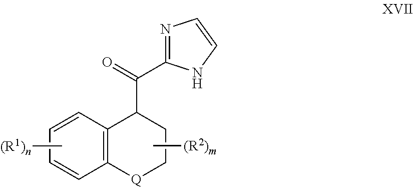

with NaOH and hydrazine hydrate
to obtain a compound of formula

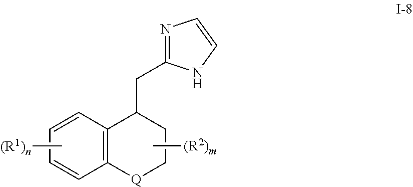

wherein R¹, R², m and n are as defined above and Q is O or CH₂, and

if desired, converting the compounds obtained into pharmaceutically acceptable acid addition salts.

Bicyclic substituted 2-imidazoline, 2-imidazole and 2-imidazole compounds of the invention were prepared in analogy to literature procedures following the pathways depicted in Schemes 1 to 6.

These procedures are described in following references
[1] J. Med. Chem. 1986, 29, 1413
[2] Bull. Korean Chem. Soc. 2003, 24, 1354
[3] J. Med. Chem. 1987, 30, 1482
[4] Chem. Pharm. Bull. 1987, 35, 1058 and Synthesis 1990, 78.
[5] J. Med. Chem. 1997, 40, 3014
[6] Tetrahedron 2004, 60, 9857
[7] Synth. Commun. 1990, 20, 2483
[8] Org. Lett. 2002, 4, 3051

All starting materials are either commercially available, are otherwise known in the chemical literature, or can be prepared in accordance with methods well known in the art.

### Procedure A

### Synthesis of Bicyclic Substituted Imidazolines

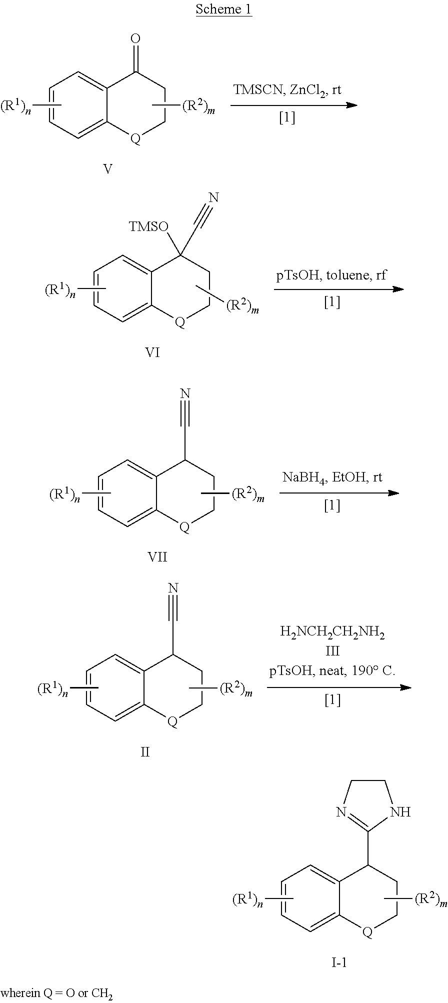

2-Imidazolines of formula I-1 can be prepared by reaction of a nitrile of formula II with ethylenediamine of formula III. This cyclization with a diamine can be conducted by heating a diamine mono p-toluenesulfonic acid salt with a nitrile neat at 100° C. to 250° C., preferably at 140° C. to 240° C., for several hours, preferably 2 to 6 hours, or by heating a solution of the nitrile in an excess of ethylenediamine or a derivative thereof in presence of a catalytic amount of sulfur, preferably 10 mol % to 50 mol %, in a sealed tube under microwave irradiation to 200° C. for 10 to 60 minutes, preferably for 15 to 30 minutes [2], or by reaction of a complex preformed from trimethylaluminum and ethylenediamine or a derivative thereof in toluene below ambient temperature, preferably at 0° C. to 10° C., with a nitrile in toluene at reflux temperature for 4 to 24 hours, preferably for 16 to 20 hours [3]. In the latter procedure the nitrile can be replaced by the corresponding lower alkyl ester.

Nitriles of formula II derived from cyclic ketones of formula V may be prepared in a three step procedure following procedures known in the literature. The sequence starts with addition of a synthetic equivalent of hydrogen cyanide, e.g. trimethylsilyl cyanide, which results in the formation of an O-protected cyanohydrin of formula VI, e.g, trimethylsilyl-0. This addition is performed in the presence of a catalyst, e.g. zinc iodide, neat at ambient temperature under vigorous stirring for 18 to 48 hours. Elimination of trimethylsilanol in the presence of a catalytic amount of an acid, preferred is p-toluenesulfonic acid, in an organic solvent like benzene, toluene, xylene and the like, preferably toluene, at reflux temperature for 1 to 6 hours, preferably 2 to 3 hours, provides the α,β-unsaturated nitrile of formula VII. Reduction of the double bond in this nitrile with a complex hydride, preferred is sodium borohydride, in a lower alcohol like methanol, ethanol, isopropanol, preferred is ethanol, at reflux temperature for 0.5 to 2 hours, preferably 0.5 to 1 hour, furnishes the nitrile of formula II.

### Procedure B

### Synthesis of Bicyclic Substituted Imidazoles

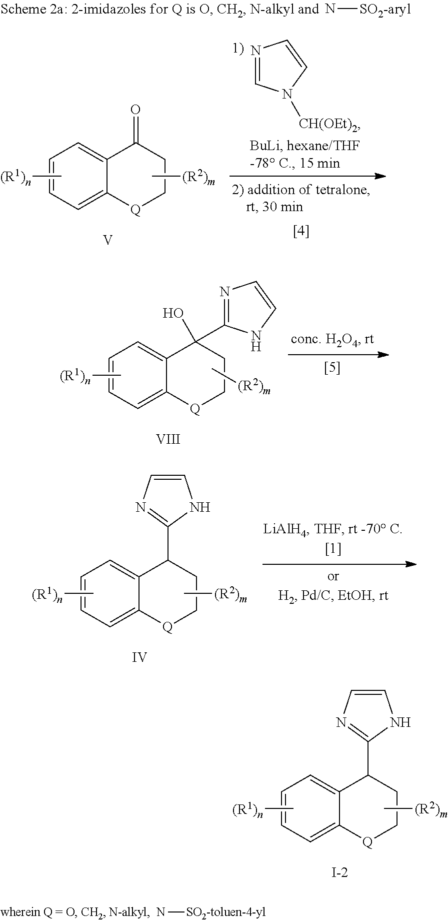

Direct introduction of the 2-imidazole residue is done by reaction of an aryl ketone V with a metallated N-protected imidazole, which is first prepared in situ by deprotonation of an N-protected imidazole with a strong base like alkyl or aryl lithium, preferably by n-butyl lithium, in an inert organic solvent, e.g. tetrahydrofuran or diethyl ether, below ambient temperature, preferably at −78° C. The primary product isolated is a tertiary alcohol of formula VIII.

The α,β-unsaturated 2-imidazoles of formula R¹ are obtained from the corresponding tertiary alcohols by acid catalyzed elimination of water. Preferred catalyst is p-toluenesulfonic acid and the reaction is run in an azeotrope-forming solvent like benzene or toluene, preferred is toluene, at reflux temperature for 1 to 4 hours, preferred are 2 to 3 hours. The reaction can also be performed by adding the corresponding tertiary alcohols to conc. sulfuric acid at 0° C. to ambient temperature, preferred is 0° C. to 10° C., and then stirring the mixture at ambient temperature for 5 to 30 minutes, preferred is 10 to 15 minutes.

The 2-imidazoles of formula I-2 are prepared from the corresponding α,β-unsaturated 2-imidazoles of formula R¹ by reduction of the double bond either by catalytic hydrogenation in the presence of Pd/C in a polar solvent, preferred is a lower alcohol, or by a complex hydride like lithium aluminum hydride in an aprotic solvent like tetrahydrofuran or diethylether at ambient temperature or elevated temperature for 2 to 12 hours, preferably 4 to 8 hours. In the case where Q is N—SO₂-aryl, reduction using lithium aluminium hydride at elevated temperature affords a mixture of the corresponding products of formula I-2 where Q is N—SO₂-aryl and Q is NH.

Formation of the latter compound is favored by extended reaction times or increased reaction temperatures.

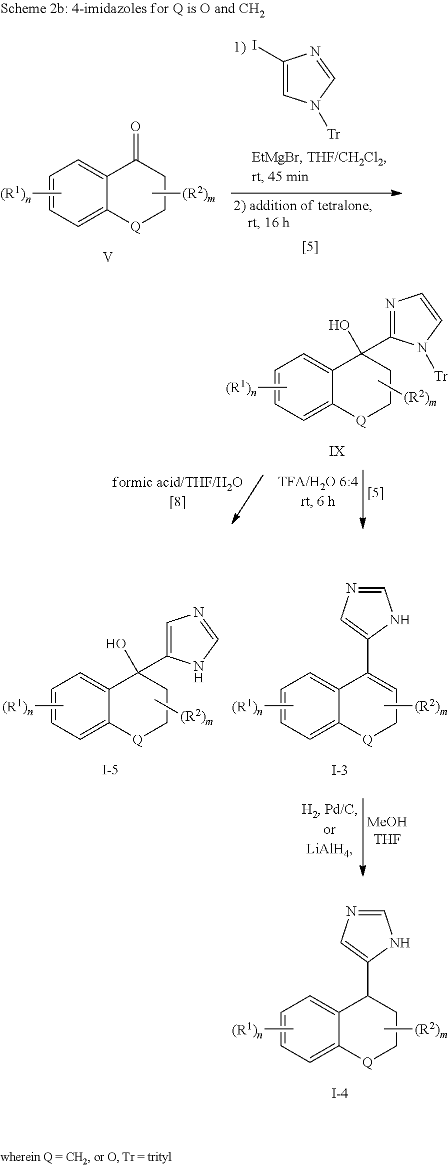

Direct introduction of the 4-imidazole residue is done by reaction of an aryl ketone of formula V with a metallated N-protected imidazole which is first generated in situ from an N-protected 4-iodo-imidazole by treatment with an organomagnesium reagent, preferably ethylmagnesium bromide, in an inert organic solvent, preferably in a mixture of dichloromethane and tetrahydrofuran, at ambient temperature. The primary product isolated is a tertiary alcohol of formula IX.

The α,β-unsaturated and N-deprotected 4-imidazoles of formula I-3 are obtained from the corresponding tertiary alcohols by acid catalyzed elimination of water as described for the 2-imidazoles. The trityl group on the imidazole is also eliminated under these reaction conditions. In addition to the procedures mentioned for the preparation of α,β-unsaturated 2-imidazoles, the reaction with 30% to 80% trifluoroacetic acid in water, preferred is 60%, at ambient temperature for 12 to 24 hours, preferred is 14 to 18 hours, also provides the α,β-unsaturated and detritylated 4-imidazoles of formula I-3.

The N-deprotected 4-imidazoles of formula I-5 still bearing the tertiary alcohol are obtained by acid catalyzed deprotection of the corresponding N-trityl-imidazole with a mixture of formic acid/THF/water 1:1:0.1.

In analogy to the 2-imidazoles the 4-imidazoles of formula I-4 are prepared from the corresponding α,β-unsaturated 4-imidazoles of formula I-3 by reduction of the double bond either by catalytic hydrogenation in the presence of Pd/C in a polar solvent like methanol, ethanol, propanol, isopropanol or ethyl acetate, preferred is a lower alcohol like methanol or ethanol, or by reduction using a complex hydride like lithium aluminum hydride in an aprotic solvent like tetrahydrofuran or diethylether at ambient temperature for 2 to 12 hours, preferably for 4 to 8 hours.

### Procedure C(C1 and C2)

### Dehydrogenation of Imidazolines to Imidazoles

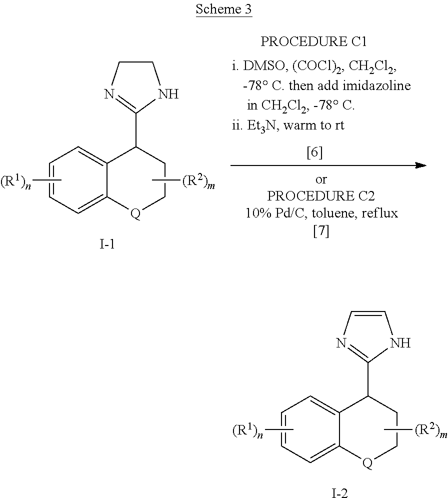

The 2-imidazoles of formula I-2 can also be prepared by dehydrogenation of the corresponding 2-imidazolines. Two procedures described in the literature have been used for this transformation, Swern type oxidation and catalytic dehydrogenation.

### Procedure D

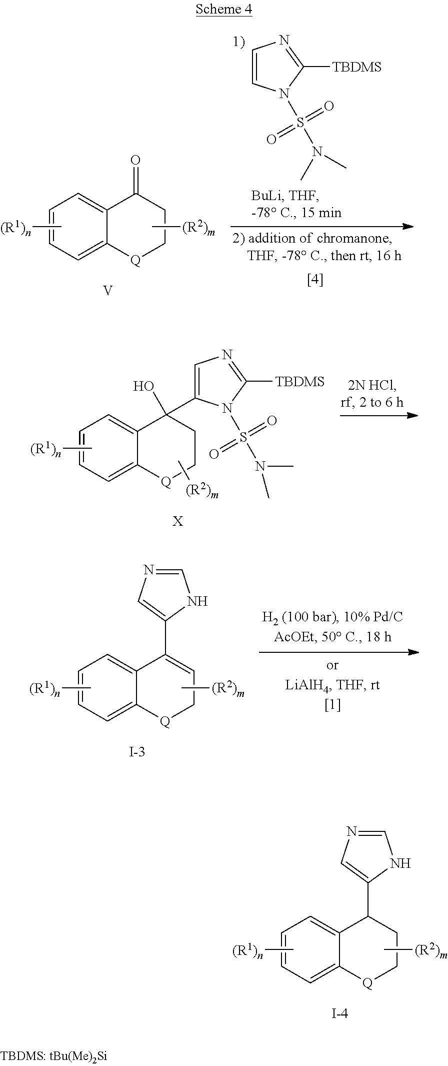

Direct introduction of the 4-imidazole residue can also be done in analogy to the procedure published by S. Ohta et al. (Synthesis 1990, 78) by reaction of an aryl ketone of formula V with a metallated N(1)-and C(2)-diprotected imidazole, preferred is 2-(tert-butyl-dimethyl-silanyl)-imidazole-1-sulfonic acid dimethylamide, which is deprotonated in situ with a strong base like alkyl or aryl lithium, preferably by n-butyl lithium, in an inert organic solvent, e.g. tetrahydrofuran or diethyl ether, below ambient temperature, preferably at −78° C. The primary product isolated is a tertiary alcohol of formula X.

Heating of a solution of the tertiary alcohol X in diluted mineral acid, preferred is 1 N to 4 N HCl, at reflux for 2 to 6 hours provides the α,β-unsaturated bicyclic product of formula I-3 bearing a deprotected 4-imidazolyl residue.

The 4-imidazoles of formula I-4 are prepared from the corresponding α,β-unsaturated 2-imidazoles of formula I-3 by reduction of the double bond either by catalytic hydrogenation with pressurized hydrogen at 50 to 150 bar, preferred is 100 bar, in the presence of Pd/C in a polar solvent like methanol, ethanol, propanol, isopropanol or ethyl acetate, preferred is ethyl acetate, at a temperature between ambient temperature and 150° C., preferred is 50° C., for 12 to 24 hours, preferred is 16 to 20 hours, or by reduction using a complex hydride like lithium aluminum hydride in an aprotic solvent like tetrahydrofuran or diethyl ether at ambient temperature for 2 to 12 hours, preferably for 4 to 8 hours.

### Procedure E

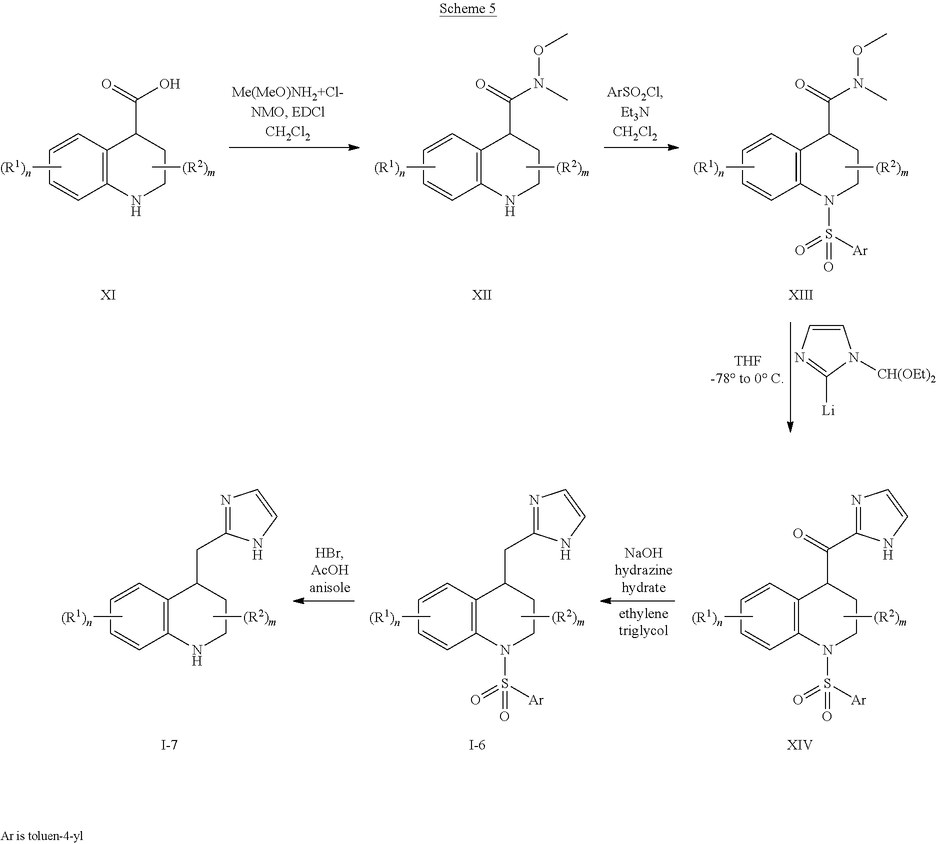

2-imidazole compounds of formula I-7 where W is CH₂ and Q is NH, N-alkyl, N—SO₂-alkyl or N—SO₂-toluen-4-yl can be prepared as shown in scheme 5. The starting materials are 1,2,3,4-tetrahydro-quinoline-4-carboxylic acid compounds of formula XI, which can be prepared by methods already reported in the literature, for instance by Raney nickel reduction of the corresponding quinoline-4-carboxylic acid compounds, as reported in Khimiya Geterotsiklicheskikh Soedinenii 1988, 77-9. The carboxylic acid compounds of formula XI are converted to the corresponding Weinreb amide derivatives of formula XII by treatment with N,O-dimethylhydroxylamine hydrochloride and a coupling reagent such as 1-ethyl-3-(3′-dimethylaminopropyl)carbodiimide hydrochloride (EDCI) in the presence of a tertiary amine base such as triethylamine or N-methylmorpholine. The reaction is carried out in a halogenated organic solvent such as dichloromethane.

Following preparation of the Weinreb amide compounds of formula XII, the nitrogen atom of the 1,2,3,4-tetrahydro-quinoline ring system is protected, for instance as the corresponding arylsulphonamide, by treatment with an arylsulphonyl chloride in the presence of a tertiary amine base such as triethylamine in a halogenated organic solvent such as dichloromethane or 1,2-dichloroethane. The reaction can be performed at room temperature or at the reflux temperature of the solvent used.

The Weinreb amide moiety present in the compounds of formula XIII can then be reacted with a metallated N-protected imidazole, for instance with 2-(1-diethoxymethyl-1H-imidazol-2-yl)-lithium, which is first prepared in situ by deprotonation of the corresponding N-protected imidazole with a strong base like alkyl or aryl lithium, preferably by n-butyl lithium, in an inert ethereal solvent, e.g. tetrahydrofuran or diethyl ether, below ambient temperature, preferably at −78° C. The reaction between the Weinreb amide compound of formula XIII and the metallated N-protected imidazole is performed in an inert ethereal solvent, e.g. tetrahydrofuran or diethyl ether, below ambient temperature, preferably at a temperature between −78° C. and 0° C. The primary product isolated is a ketone of formula XIV.

The ketone of formula XIV can then be subjected to Wolff-Kischner reduction to afford a compound of formula I-6, for instance using the procedure reported in Arch. Pharm. 1989, 322, 363-367 which involves treatment with sodium hydroxide and hydrazine hydrate in a high boiling point organic solvent such as triethylene glycol, at elevated temperature, preferably at temperatures between 110° C. and 200° C.

Finally, the protecting group in the compound of formula I-6 can be removed, for instance by reaction with a protic acid such as HBr in acetic acid in the presence of anisole, to afford the desired compounds of formula I-7.

### Procedure F

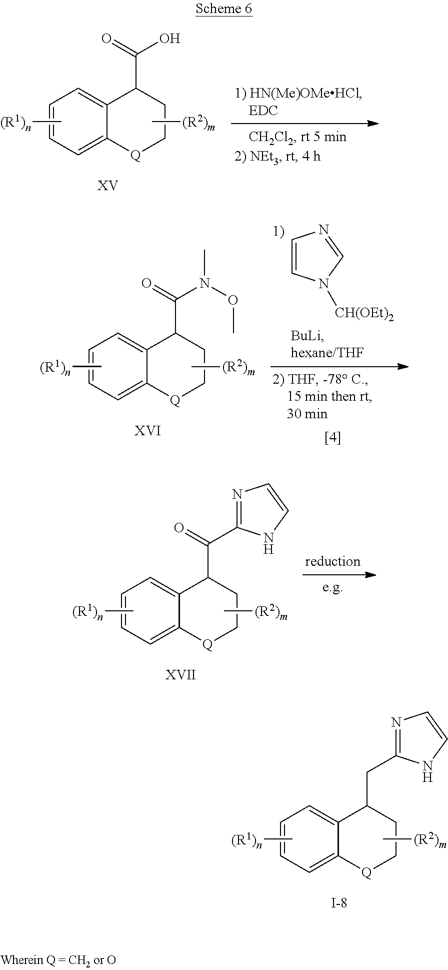

The starting material, a Weinreb type amide of formula XVI, is prepared from the corresponding carboxylic acid of formula XV following procedures known in the art (cf. Scheme 5). Direct introduction of the 2-imidazole residue is done by reaction of the Weinreb type amide with a metallated N-protected imidazole which is generated in situ from an N-protected-imidazole with a strong base like alkyl or aryl lithium, preferably n-butyl lithium, in an inert organic solvent, e.g. tetrahydrofuran or diethyl ether, below ambient temperature, preferably at −78° C. The primary product isolated is a ketone of formula XVII.

Reduction of this ketone following procedures known in the art, e.g. a Wolff-Kishner type reduction (cf. Scheme 5), provides the final product of formula I-8.

The corresponding 4-imidazoles are accessible following the route depicted in Scheme 4 by using the 1,2-diprotected imidazoles shown in Scheme 4.

Compounds of formula I, wherein R is tritium can be prepared from the corresponding halogenated (chloro, bromo or iodo) compound, preferred is the bromo-substituted compound, by catalytic hydrogenation with tritium gas.

### Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula I can be separated using chiral HPLC.

### Salts of Compounds of Formula I

The compounds of formula I are basic and can be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or can be brought out of solution with a less polar solvent.

The acid addition salts of the basic compounds of formula I can be converted to the corresponding free bases by treatment with at least a stoichiometric equivalent of a suitable base such as sodium or potassium hydroxide, potassium carbonate, sodium bicarbonate, ammonia, and the like.

The compounds of formula I and their pharmaceutically acceptable addition salts possess valuable pharmacological properties. Specifically, compounds of the present invention have a good affinity to the trace amine associated receptors (TAARs), especially TAAR1.

The compounds were investigated in accordance with the test given hereinafter.

### Materials and Methods

### Construction of TAAR Expression Plasmids and Stably Transfected Cell Lines

For the construction of expression plasmids the coding sequences of human, rat and mouse TAAR 1 were amplified from genomic DNA essentially as described by Lindemann et al. [14]. The Expand High Fidelity PCR System (Roche Diagnostics) was used with 1.5 mM Mg²⁺ and purified PCR products were cloned into pCR2.1-TOPO cloning vector (Invitrogen) following the instructions of the manufacturer. PCR products were subcloned into the pIRESneo2 vector (BD Clontech, Palo Alto, Calif.), and expression vectors were sequence verified before introduction in cell lines.

HEK293 cells (ATCC # CRL-1573) were cultured essentially as described Lindemann et al. (2005). For the generation of stably transfected cell lines HEK293 cells were transfected with the pIRESneo2 expression plasmids containing the TAAR coding sequences (described above) with Lipofectamine 2000 (Invitrogen) according to the instructions of the manufacturer, and 24 hrs post transfection the culture medium was supplemented with 1 mg/ml G418 (Sigma, Buchs, Switzerland). After a culture period of about 10 d clones were isolated, expanded and tested for responsiveness to trace amines (all compounds purchased from Sigma) with the cAMP Biotrak Enzyme immunoassay (EIA) System (Amersham) following the non-acetylation EIA procedure provided by the manufacturer. Monoclonal cell lines which displayed a stable EC₅₀ for a culture period of 15 passages were used for all subsequent studies.

### Membrane Preparation and Radioligand Binding

Cells at confluence were rinsed with ice-cold phosphate buffered saline without Ca²⁺ and Mg²⁺ containing 10 mM EDTA and pelleted by centrifugation at 1000 rpm for 5 min at 4° C. The pellet was then washed twice with ice-cold phosphate buffered saline and cell pellet was frozen immediately by immersion in liquid nitrogen and stored until use at −80° C. Cell pellet was then suspended in 20 ml HEPES-NaOH (20 mM), pH 7.4 containing 10 mM EDTA, and homogenized with a Polytron (PT 3000, Kinematica) at 10,000 rpm for 10 s. The homogenate was centrifuged at 48,000×g for 30 min at 4° C. and the pellet resuspended in 20 ml HEPES-NaOH (20 mM), pH 7.4 containing 0.1 mM EDTA (buffer A), and homogenized with a Polytron at 10,000 rpm for 10 s. The homogenate was then centrifuged at 48,000×g for 30 min at 4° C. and the pellet resuspended in 20 ml buffer A, and homogenized with a Polytron at 10,000 rpm for 10 s. Protein concentration was determined by the method of Pierce (Rockford, Ill.). The homogenate was then centrifuged at 48,000×g for 10 min at 4° C., resuspended in HEPES-NaOH (20 mM), pH 7.0 including MgCl₂ (10 mM) and CaCl₂ g protein per ml and (2 mM) (buffer B) at 200 homogenized with a Polytron at 10,000 rpm for 10 s.

Binding assay was performed at 4° C. in a final volume of 1 ml, and with an incubation time of 30 min. The radioligand [³H]-rac-2-(1,2,3,4-tetrahydro-1-naphthyl)-2-imidazoline was used at a concentration equal to the calculated K_{d} value of 60 nM to give a bound at around 0.1% of the total added radioligand concentration, and a specific binding which represented approximately 70-80% of the total binding. Non-specific binding was defined as the amount of [³H]-rac-2-(1,2,3,4-tetrahydro-1-naphthyl)-2-imidazoline bound in the presence of the appropriate unlabelled ligand (10 μM). Competing ligands were tested in a wide range of concentrations (10 pM-30 μM). The final dimethylsulphoxide concentration in the assay was 2%, and it did not affect radioligand binding. Each experiment was performed in duplicate. All incubations were terminated by rapid filtration through UniFilter-96 plates (Packard Instrument Company) and glass filter GF/C, pre-soaked for at least 2 h in polyethylenimine 0.3%, and using a Filtermate 96 Cell Harvester (Packard Instrument Company). The tubes and filters were then washed 3 times with 1 ml aliquots of cold buffer B. Filters were not dried and soaked in Ultima gold (45 μl/well, Packard Instrument Company) and bound radioactivity was counted by a TopCount Microplate Scintillation Counter (Packard Instrument Company).

The preferred compounds show a Ki value (μM) on mouse TAAR1 in the range of 0.009-0.060. Representative compounds are provided in the table below.

| Example | |
|---|---|
| | Ki (μM) |
| | mouse |
| 1 | 0.009 |
| 2 | 0.011 |
| 3 | 0.054 |
| 10 | 0.013 |
| 11 | 0.017 |
| 12 | 0.054 |
| 18 | 0.058 |
| 19 | 0.013 |
| 20 | 0.051 |
| 21 | 0.009 |
| 22 | 0.025 |
| 36 | 0.053 |
| | Ki |
| 41 | 0.061 |
| 55 | 0.011 |
| 56 | 0.006 |
| 60 | 0.008 |
| 62 | 0.004 |
| 63 | 0.020 |
| 64 | 0.016 |
| 65 | 0.004 |
| 66 | 0.037 |
| 67 | 0.032 |
| 69 | 0.025 |

The present invention also provides pharmaceutical compositions containing compounds of the invention, for example compounds of formula I and their pharmaceutically suitable acid addition salts, and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compounds of the invention, in addition to one or more compounds of the invention, contain a pharmaceutically acceptable carrier. Suitable pharmaceutically acceptable carriers include pharmaceutically inert, inorganic and organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The invention also provides a method for preparing compositions of the invention which comprises bringing one or more compounds of formula I and/or pharmaceutically acceptable acid addition salts and, if desired, one or more other therapeutically valuable substances into a galenical administration form together with one or more therapeutically inert carriers.

The most preferred indications in accordance with the present invention are those, which include disorders of the central nervous system, for example the treatment or prevention of depression, psychosis, Parkinson's disease, anxiety and attention deficit hyperactivity disorder (ADHD). Thus, the invention provides a method for the treatment of depression, which comprises administering to an individual a therapeutically effective amount of a compound of the invention and a pharmaceutically acceptable carrier. The invention also provides a method for the treatment of psychosis, which comprises administering to an individual a therapeutically effective amount of a compound of the invention and a pharmaceutically acceptable carrier. The invention further provides a method for the treatment of Parkinson's disease, which comprises administering to an individual a therapeutically effective amount of a compound of the invention and a pharmaceutically acceptable carrier. The invention provides a method for the treatment of anxiety, which comprises administering to an individual a therapeutically effective amount of a compound of the invention and a pharmaceutically acceptable carrier. The invention also provides a method for the treatment of attention deficit hyperactivity disorder (ADHD), which comprises administering to an individual a therapeutically effective amount of a compound of the invention and a pharmaceutically acceptable carrier.

The compounds and compositions of the present invention can be administered in a conventional manner, for example, orally, rectally, or parenterally. The compounds of the invention can be administered orally, for example, in the form of tablets, coated tablets, dragées, hard and soft gelatin capsules, solutions, emulsions, or suspensions. The compounds of the invention can be administered rectally, for example, in the form of suppositories or parenterally, for example, in the form of injectable solutions.

The dosage at which compounds of the invention can be administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

Manufacturing Procedure
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

| | | Capsule Formulation | | | |
|---|---|---|---|---|---|
| | | mg/capsule | | | |
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Manufacturing Procedure
1. Mix items 1, 2 and 3 in a suitable mixer for 30 minutes.
2. Add items 4 and 5 and mix for 3 minutes.
3. Fill into a suitable capsule.

### EXPERIMENTAL

The following examples illustrate the invention but are not intended to limit its scope.

### Procedure A

### Example 1

### rac-2-(5-Bromo-1,2,3,4-tetrahydro-naphthalen-1-yl)-4,5-dihydro-1H-imidazole

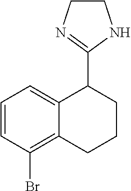

A mixture of 400 mg (1.7 mmol) rac-5-bromo-1,2,3,4-tetrahydro-naphthalene-1-carbonitrile and 511 mg (2.2 mmol) ethylene diamine p-toluenesulfonic acid mono salt was heated neat to 150° C. and the liquid stirred for 6 hours at this temperature. Then the cooled reaction mixture was diluted with water and saturated aqueous solution of potassium carbonate. The solution was extracted with ethyl acetate, the combined extracts washed with brine, dried over Na₂SO₄, filtered and evaporated. Purification of the crude product by flash-chromatography over silica gel with methanol/concentrated ammonia 98:2 as eluent provided pure rac-2-(5-bromo-1,2,3,4-tetrahydro-naphthalen-1-yl)-4,5-dihydro-1H-im as colorless solid; MS (ISP): 281.0 and 279.0 ((M+H)₊.).

### Example 2

### rac-2-(5,7-Dimethyl-1,2,3,4-tetrahydro-naphthalen-1-yl)-4,5-dihydro-1H-imidazole

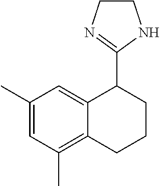

rac-2-(5,7-Dimethyl-1,2,3,4-tetrahydro-naphthalen-1-yl)-4,5-dihydro-1H-imidazole was prepared from rac-5,7-dimethyl-1,2,3,4-tetrahydro-naphthalene-1-carbonitrile in analogy to Example 1: colorless solid; MS (EI): 228.3 (M⁺.).

### Example 3

### rac-2-(7-Chloro-5-fluoro-1,2,3,4-tetrahydro-naphthalen-1-yl)-4,5-dihydro-1H-imidazole

### a) rac-7-Chloro-5-fluoro-1-trimethylsilanyloxy-1,2,3,4-tetrahydro-naphthalene-1-carbonitrile

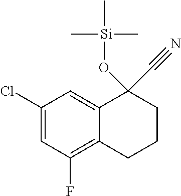

To 2.00 g (11.3 mmol) 7-chloro-5-fluoro-3,4-dihydro-2H-naphthalen-1-one were added 0.11 g (0.35 mmol) zinc iodide and under vigorous stirring 3.72 g (4.69 ml, 37.4 mmol) trimethylsilyl cyanide drop-wise over 15 min. The mixture was stirred at ambient temperature over night, and then diluted with ethyl acetate. The organic phase was washed twice with saturated aqueous sodium bicarbonate solution, brine, dried over Na₂SO₄, filtered and evaporated. The crude product was filtered through a silica gel pad with heptane/ethyl acetate 4:1 as eluent: rac-7-Chloro-5-fluoro-1-trimethylsilanyloxy-1,2,3,4-tetrahydro-naphthalene-1-carbonitrile was obtained as a light yellow liquid: MS (EI): 297.2 (M⁺.), 282.2 ((M−CH₃)⁺.), 271.2 ((M−CN)⁺.), 255.1 (((M−(CH₃+HCN))⁺., 100%), 207.1 ((M−(CH₃)₃SiOH)⁺.).

### b) 7-Chloro-5-fluoro-3,4-dihydro-naphthalene-1-carbonitrile

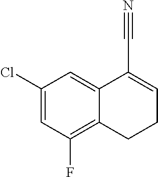

To 4.5 ml concentrated (96%) sulfuric acid cooled to 0° C. were added under vigorous stirring 1.00 g (3.4 mmol) rac-7-chloro-5-fluoro-1-trimethylsilanyloxy-1,2,3,4-tetrahydro-naphthalene-1-carbonitrile drop-wise over 5 min. Then the cooling bath was removed and the mixture stirred for 10 min. Then ice was added and the mixture made alkaline by addition of concentrated aqueous sodium hydroxide solution. The aqueous solution was extracted with dichloromethane, the combined organic extracts washed with brine, dried over Na₂SO₄, filtered and evaporated. The crude product was filtered through a silica gel with heptane/ethyl acetate 1:1 as eluent: 0.63 g (90%) 7-chloro-5-fluoro-3,4-dihydro-naphthalene-1-carbonitrile.

### c) rac-7-Chloro-5-fluoro-1,2,3,4-tetrahydro-naphthalene-1-carbonitrile

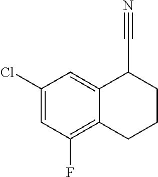

To a solution of 300 mg (1.44 mmol) 7-chloro-5-fluoro-3,4-dihydro-naphthalene-1-carbonitrile in 4 ml ethanol were added 328 mg (8.67 mmol) sodium borohydride and the mixture was heated to reflux for 30 min. The reaction mixture was cooled down and concentrated. The residue was distributed between water and dichloromethane. The combined organic extracts were washed with brine, dried over Na₂SO₄, filtered and evaporated. The crude product was purified by flash-chromatography with a heptane/ethyl acetate gradient as eluent. rac-7-Chloro-5-fluoro-1,2,3,4-tetrahydro-naphthalene-1-carbonitrile was obtained as colorless oil: MS (EI): 209.2 (M⁺.), 182.1 ((M−HCN)⁺.), 156.1 ((M−CH₂═CHCN)⁺.), 147.2 (((M−(Cl+HCN))⁺.), 100%).

### d) rac-2-(7-Chloro-5-fluoro-1,2,3,4-tetrahydro-naphthalen-1-yl)-4,5-dihydro-1H-imidazole

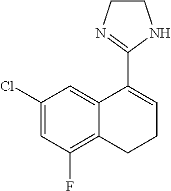

rac-2-(7-Chloro-5-fluoro-1,2,3,4-tetrahydro-naphthalen-1-yl)-4,5-dihydro-1H-imidazole was prepared from rac-7-chloro-5-fluoro-1,2,3,4-tetrahydro-naphthalene-1-carbonitrile in analogy to Example 1 but heated to 240° C. for 2 hours: colorless crystalline solid; MS (ISP): 253.1 ((M+H)⁺.).

### Example 4

### rac-2-(7-Fluoro-1,2,3,4-tetrahydro-naphthalen-1-yl)-4,5-dihydro-1H-imidazole

### a) rac-7-Fluoro-1-trimethylsilanyloxy-1,2,3,4-tetrahydro-naphthalene-1-carbonitrile

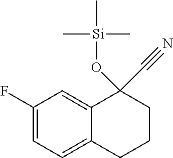

rac-7-Fluoro-1-trimethylsilanyloxy-1,2,3,4-tetrahydro-naphthalene-1-carbonitrile was prepared from 7-fluoro-3,4-dihydro-2H-naphthalen-1-one in analogy to Example 3 a): light yellow liquid; MS (EI): 263.2 (M⁺.), 248.2 ((M−CH₃)⁺.), 237.2 ((M−CN)⁺.), 221.2 ((M−(CH₃+HCN))⁺.), 173.2 (((M−(CH₃)₃SiOH)⁺.), 100%).

### b) 7-Fluoro-3,4-dihydro-naphthalene-1-carbonitrile

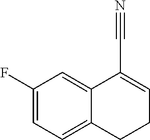

7-Fluoro-3,4-dihydro-naphthalene-1-carbonitrile was prepared from rac-7-fluoro-1-trimethyl-silanyloxy-1,2,3,4-tetrahydro-naphthalene-1-carbonitrile in analogy to Example 3b).

### c) rac-7-Fluoro-1,2,3,4-tetrahydro-naphthalene-1-carbonitrile

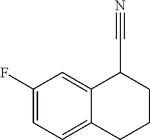

rac-7-Fluoro-1,2,3,4-tetrahydro-naphthalene-1-carbonitrile was prepared from 7-fluoro-3,4-dihydro-naphthalene-1-carbonitrile in analogy to Example 3c): light yellow liquid; MS (EI): 175.2 (M⁺.), 148.2 (((M−HCN)⁺.), 100%), 122.1 ((M−CH₂═CHCN)⁺.).

### d) rac-2-(7-Fluoro-1,2,3,4-tetrahydro-naphthalen-1-yl)-4,5-dihydro-1H-imidazole

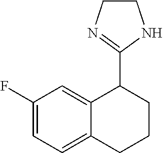

rac-2-(7-Fluoro-1,2,3,4-tetrahydro-naphthalen-1-yl)-4,5-dihydro-1H-imidazole was prepared from rac-7-fluoro-1,2,3,4-tetrahydro-naphthalene-1-carbonitrile in analogy to Example 3d): light yellow solid; MS (EI): 218.2 (M⁺.).

### Example 5

### rac-2-(8-Methoxy-1,2,3,4-tetrahydro-naphthalen-1-yl)-4,5-dihydro-1H-imidazole

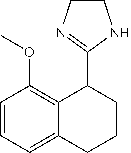

rac-2-(8-Methoxy-1,2,3,4-tetrahydro-naphthalen-1-yl)-4,5-dihydro-1H-imidazole was prepared from rac-8-Methoxy-1,2,3,4-tetrahydro-naphthalene-1-carbonitrile in analogy to Example 3d): light yellow gum; MS (ISP): 231.2 ((M+H)⁺.).

### Example 6

### rac-2-(7-Bromo-1,2,3,4-tetrahydro-naphthalen-1-yl)-4,5-dihydro-1H-imidazole

### a) rac-7-Bromo-1-trimethylsilanyloxy-1,2,3,4-tetrahydro-naphthalene-1-carbonitrile

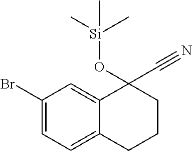

rac-7-Bromo-1-trimethylsilanyloxy-1,2,3,4-tetrahydro-naphthalene-1-carbonitrile was prepared from 7-bromo-3,4-dihydro-2H-naphthalen-1-one in analogy to Example 3 a): colorless solid, m.p. 45-47° C.; MS (EI): 325.1 and 323.1 (M⁺.), 310.1 and 308.1 ((M−CH₃)⁺.), 283.1 and 281.0 ((M−(CH₃+HCN))⁺.), 235.1 and 233.1 (((M−(CH₃)₃SiOH)⁺.), 100%), 202.2 ((M−(CH₃+HCN+Br))⁺.).

### b) 7-Bromo-3,4-dihydro-naphthalene-1-carbonitrile

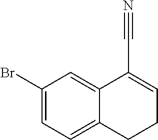

7-Bromo-3,4-dihydro-naphthalene-1-carbonitrile was prepared from rac-7-bromo-1-trimethylsilanyloxy-1,2,3,4-tetrahydro-naphthalene-1-carbonitrile in analogy to Example 3b): colorless solid, m.p. 113-115° C.; ¹H-NMR (CDCl₃): 2.48-2.55 m, 2H(═CH—CH₂), 2.81 t, J=8.1 Hz, 2H(CH₂-aryl), 6.94 t, J=4.2 Hz, 1H (═CH), 7.03 d, J=7.8 Hz, 1H, and 7.38 dd, J=7.8 and 1.8 Hz, 1H, and 7.59 d, J=1.8 Hz, 1H (aryl-H).

### c) rac-7-Bromo-1,2,3,4-tetrahydro-naphthalene-1-carbonitrile

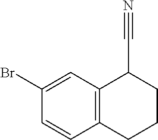

rac-7-Bromo-1,2,3,4-tetrahydro-naphthalene-1-carbonitrile was prepared from 7-bromo-3,4-dihydro-naphthalene-1-carbonitrile in analogy to Example 3c): colorless oil; MS (EI): 236.0 and 234.9 (M⁺.), 210.0 and 207.9 ((M−HCN)⁺.), 129.0 (((M−(HCN+Br))⁺.), 100%).

### d) rac-2-(7-Bromo-1,2,3,4-tetrahydro-naphthalen-1-yl)-4,5-dihydro-1H-imidazole

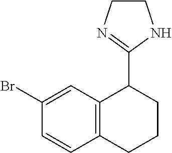

rac-2-(7-Bromo-1,2,3,4-tetrahydro-naphthalen-1-yl)-4,5-dihydro-1H-imidazole was prepared from rac-7-bromo-1,2,3,4-tetrahydro-naphthalene-1-carbonitrile in analogy to Example 1 but heated to 210° C. for 2 hours: colorless solid, m.p. 156-158° C.; MS (ISP): 281.1 and 279.0 ((M+H)⁺.).

### Example 7

### rac-2-(5,7-Dibromo-1,2,3,4-tetrahydro-naphthalen-1-yl)-4,5-dihydro-1H-imidazole

### a) rac-5,7-Dibromo-1-trimethylsilanyloxy-1,2,3,4-tetrahydro-naphthalene-1-carbonitrile

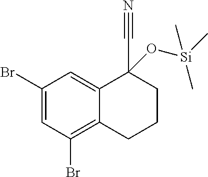

rac-5,7-Dibromo-1-trimethylsilanyloxy-1,2,3,4-tetrahydro-naphthalene-1-carbonitrile was prepared from 5,7-dibromo-3,4-dihydro-2H-naphthalen-1-one in analogy to Example 3 a): grey solid.

### b) 5,7-Dibromo-3,4-dihydro-naphthalene-1-carbonitrile

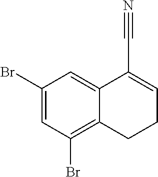

5,7-Dibromo-3,4-dihydro-naphthalene-1-carbonitrile was prepared from rac-5,7-dibromo-1-trimethylsilanyloxy-1,2,3,4-tetrahydro-naphthalene-1-carbonitrile in analogy to Example 3b): off-white solid.

### c) rac-5,7-Dibromo-1,2,3,4-tetrahydro-naphthalene-1-carbonitrile

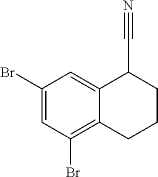

rac-5,7-Dibromo-1,2,3,4-tetrahydro-naphthalene-1-carbonitrile was prepared from 5,7-dibromo-3,4-dihydro-naphthalene-1-carbonitrile in analogy to Example 3c): colorless liquid.

### d) rac-2-(5,7-Dibromo-1,2,3,4-tetrahydro-naphthalen-1-yl)-4,5-dihydro-1H-imidazole

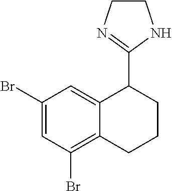

rac-2-(5,7-Dibromo-1,2,3,4-tetrahydro-naphthalen-1-yl)-4,5-dihydro-1H-imidazole was prepared from rac-5,7-dibromo-1,2,3,4-tetrahydro-naphthalene-1-carbonitrile in analogy to Example 1 but heated to 210° C. for 2 hours: light brown solid; MS (EI): 360.0 and 358.0 (100%) and 356.0 (M⁺.).

### Example 8

### rac-4-Methyl-2-(1,2,3,4-tetrahydro-naphthalen-1-yl)-4,5-dihydro-1H-imidazole or tautomer

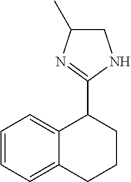

rac-4-Methyl-2-(1,2,3,4-tetrahydro-naphthalen-1-yl)-4,5-dihydro-1H-imidazole was prepared from 1,2,3,4-tetrahydro-naphthalene-1-carboxylic acid methyl ester and the complex of trimethylaluminum with 1,2-diaminopropane in toluene at reflux for 1 hour in analogy to [3]: orange gum; MS (ISP): 215.1 ((M+H)⁺.).

### Procedure C1

### Example 9

### rac-2-(1,2,3,4-Tetrahydro-naphthalen-1-yl)-1H-imidazole

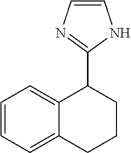

To a solution of 390 mg (0.354 ml, 5 mmol) dimethylsulfoxide in 20 ml dichloromethane cooled to −78° C. was added a solution of 634 mg (0.422 ml, 5 mmol) oxalyl chloride in 20 ml dichloromethane. The mixture was stirred for 30 minutes at −78° C. and then a solution of 200 mg (2 mmol) rac-2-(1,2,3,4-tetrahydro-1-naphthyl)-2-imidazoline in 20 ml dichloromethane was added and stirring continued at −78° C. for 1 hour. Then 1.01 g (1.4 ml) triethylamine were added and the reaction mixture warmed to ambient temperature and stirring continued for 20 minutes. Concentrated ammonia was added and the reaction mixture extracted with dichloromethane, the combined extracts washed with brine, dried over Na₂SO₄, filtered and evaporated. Purification on silica gel by flash-chromatography with a heptane/ethyl acetate gradient provided 71 mg rac-2-(1,2,3,4-tetrahydro-naphthalen-1-yl)-1H-imidazole as colorless solid: MS (EI): 198.1 (M⁺.).

### Procedure C2

### Example 10

### rac-2-(5,7-Dimethyl-1,2,3,4-tetrahydro-naphthalen-1-yl)-1H-imidazole

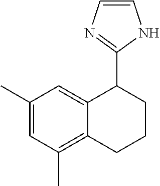

A mixture of 57 mg (0.25 mmol) rac-2-(5,7-dimethyl-1,2,3,4-tetrahydro-naphthalen-1-yl)-4,5-dihydro-1H-imidazole and 57 mg 10% Pd on charcoal in 10 ml toluene were heated to reflux for 40 hours. Then additional 30 mg 10% Pd on charcoal were added and heating to reflux continued for another 24 hours. This was repeated after 8 hours and 16 hours. After totally 88 hours at reflux temperature the reaction mixture was cooled down, filtered through a silica gel pad: 28 mg of a brown oil which was purified by flash-chromatography on silica gel with ethyl acetate as eluent. rac-2-(5,7-Dimethyl-1,2,3,4-tetrahydro-naphthalen-1-yl)-1H-imidazole was obtained as colorless crystalline solid, m.p. 161-163° C.; MS (EI): 226.3 (M⁺.).

### Example 11

### Procedure A

### rac-2-Chroman-4-yl-4,5-dihydro-1H-imidazole

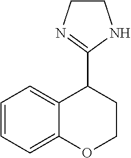

rac-2-Chroman-4-yl-4,5-dihydro-1H-imidazole was prepared from rac-chroman-4-carbonitrile in analogy to Example 1 but heated to 210° C. for 2 hours: colorless solid; MS (ISP): 202.8 ((M+H)⁺.).

### Procedure B

### Example 12

### rac-2-Chroman-4-yl-1H-imidazole

### a) rac-4-(1H-Imidazol-2-yl)-chroman-4-ol

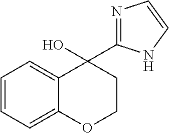

rac-4-(1H-Imidazol-2-yl)-chroman-4-ol was prepared from 4-chromanone and 2-(1-diethoxymethyl-1H-imidazol-2-yl)-lithium (prepared in situ from 1-(diethoxy-methyl)imidazole by treatment with butyl lithium in tetrahydrofuran at −78° C.) following Ohta's procedure (Synthesis 1990, 78): colorless solid; MS (EI): 216.2 (M⁺.), 95.1 (((O═C-2-imidazole)⁺.), 100%).

### b) 2-(2H-Chromen-4-yl)-1H-imidazole

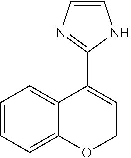

2-(2H-Chromen-4-yl)-1H-imidazole was prepared from rac-4-(1H-Imidazol-2-yl)-chroman-4-ol in analogy to Example 3b) but temperature was kept at 0° C.: light green solid: MS (ISP): 199.1 ((M+H)⁺.).

### c) rac-2-Chroman-4-yl-1H-imidazole

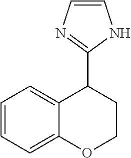

To a solution of 100 mg (0.50 mmol) 2-(2H-chromen-4-yl)-1H-imidazole in 5 ml tetrahydrofuran were added 2.02 ml of a 1M solution of lithium aluminium hydride in tetrahydrofuran and the mixture heated to reflux for 2 hours. Then the reaction mixture was cooled down to ambient temperature and the reaction quenched by slow addition of isopropanol. Water was added and the mixture was extracted with tert-butyl methyl ether, the combined organic phase washed with brine, dried over Na₂SO₄, filtered and evaporated. Purification of the crude product by flash-chromatography over a Si—NH₂ column with ethyl acetate as eluent provided rac-2-chroman-4-yl-1H-imidazole as colorless solid; MS (EI): 200.1 (M⁺.), 185.1 (((M−CH₃)⁺.), 100%).

In analogy to Example 12, Example 13 was prepared.

### Example 13

### rac-2-(6-Fluoro-chroman-4-yl)-1H-imidazole

### a) rac-6-Fluoro-4-(1H-imidazol-2-yl)-chroman-4-ol

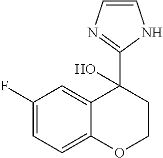

rac-6-Fluoro-4-(1H-imidazol-2-yl)-chroman-4-ol was prepared from 6-fluoro-chroman-4-one in analogy to Example 12 a): colorless solid; MS (ISP): 234.9 ((M+H)⁺.).

### b) 2-(6-Fluoro-2H-chromen-4-yl)-1H-imidazole

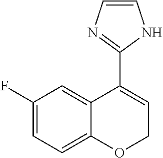

2-(6-Fluoro-2H-chromen-4-yl)-1H-imidazole was prepared from rac-6-fluoro-4-(1H-imidazol-2-yl)-chroman-4-ol in analogy to Example 12b): colorless solid: MS (EI): 216.2 (M⁺.).

### c) rac-2-(6-Fluoro-chroman-4-yl)-1H-imidazole

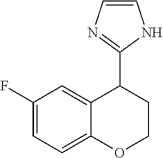

rac-2-(6-Fluoro-chroman-4-yl)-1H-imidazole was prepared from 2-(6-fluoro-2H-chromen-4-yl)-1H-imidazole in analogy to Example 12c): colorless solid: MS (EI): 218.2 (M⁺.), 203.2 (((M−CH₃)⁺.), 100%).

### Tritium Labeled Compounds

### Synthesis of [³H]-Labeled Compounds by Pd-Catalyzed Tritio-Dehalogenation Reaction of Brominated Precursors with Tritium Gas

### General Procedure

A 2 ml reaction flask containing a solution of 25-50 μmol of the brominated precursor, 15-20 mg of Pd/C (10%) and 6-10 μl of triethylamine in 1 ml of methanol was connected to the tritium manifold system (RC TRITEC AG, Teufen, Switzerland). The reaction vessel and its contents were degassed by freeze-thaw evacuation cycle and then exposed to 10-18 Ci of carrier-free tritium gas. Stirring was continued for 2-5 h at room temperature.

The solution was evaporated in vacuo and any exchangeable tritium was removed by repeated lyophilization from 3×1 ml of methanol. The residue was dissolved in 1-2 ml of ethanol and filtered through a PTFE syringe filter (0.2 μm) to remove the catalyst. After rinsing the filter with 4-8 ml of ethanol the solvent was evaporated, the residue dissolved in methanol and purified subsequently by HPLC on a standard C-18 or C-8 column.

### Example 14

### rac-2-(7-Tritio-1,2,3,4-tetrahydro-naphthalen-1-yl)-4,5-dihydro-1H-imidazole

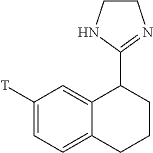

rac-2-(7-Tritio-1,2,3,4-tetrahydro-naphthalen-1-yl)-4,5-dihydro-1H-imidazole was prepared from rac-2-(7-Bromo-1,2,3,4-tetrahydro-naphthalen-1-yl)-4,5-dihydro-1H-imidazole by catalytic hydrogenation with tritium gas: >98% radiochemical purity, specific activity 32 Ci/mmol.

### Known Compounds:

| Example | | |
|---|---|---|
| No. | Structure | Name |
| 15 | 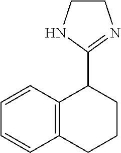 | rac-2-(1,2,3,4- tetrahydro-1- naphthyl)-2- imidazoline |
| 16 | 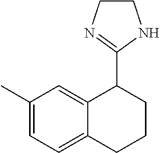 | rac-2-(7-methyl- 1,2,3,4-tetrahydro- naphthalen-1-yl)- 4,5-dihydro-1H- imidazole |
| 17 | 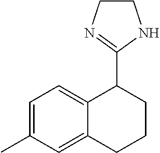 | rac-2-(6-methyl- 1,2,3,4-tetrahydro- naphthalen-1-yl)- 4,5-dihydro-1H- imidazole |
| 18 | 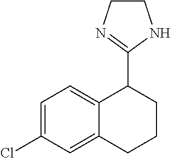 | rac-2-(6-chloro- 1,2,3,4-tetrahydro- naphthalen-1-yl)- 4,5-dihydro-1H- imidazole |
| 19 | 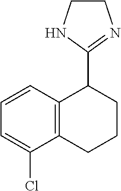 | rac-2-(5-chloro- 1,2,3,4-tetrahydro- naphthalen-1-yl)- 4,5-dihydro-1H- imidazole |
| 20 | 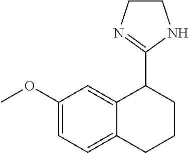 | rac-2-(7-Methoxy- 1,2,3,4-tetrahydro- naphthalen-1-yl)- 4,5-dihydro-1H- imidazole |
| 21 | 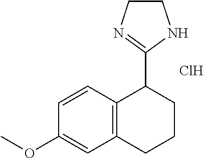 | rac-2-(6-methoxy- 1,2,3,4-tetrahydro- naphthalen-1-yl)- 4,5-dihydro-1H- imidazole |
| 22 | 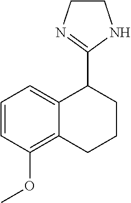 | rac-2-(5-methoxy- 1,2,3,4-tetrahydro- naphthalen-1-yl)- 4,5-dihydro-1H- imidazole |
| 23 | 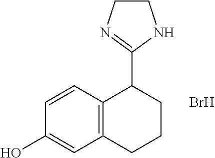 | rac-5-(4,5-dihydro- 1H-imidazol-2-yl)- 5,6,7,8-tetrahydro- naphthalen-2-ol |
| 24 | 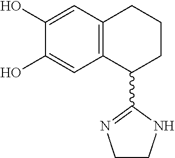 | rac-5-(4,5-Dihydro- 1H-imidazol-2-yl)- 5,6,7,8-tetrahydro- naphthalene-2,3-diol |
| 25 | 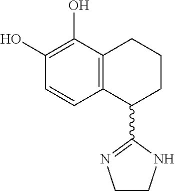 | rac-5-(4,5-Dihydro-1H- imidazol-2-yl)-5,6,7,8- tetrahydro-naphthalene- 1,2-diol |
| 56 | 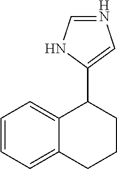 | rac-4-(1,2,3,4- Tetrahydro- naphthalen-1-yl)- 1H-imidazole |

### Example 26

### rac-2-(5-Nitro-1,2,3,4-tetrahydro-naphthalen-1-yl)-4,5-dihydro-1H-imidazole hydrochloride

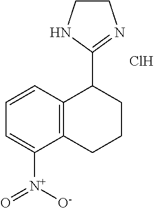

rac-2-(5-Nitro-1,2,3,4-tetrahydro-naphthalen-1-yl)-4,5-dihydro-1H-imidazole was prepared from 5-nitro-3,4-dihydro-2H-naphthalen-1-one in analogy to Example 3: colorless solid; MS (ISP): 246.1 ((M+H)⁺.).

### Example 27

### rac-8-(4,5-Dihydro-1H-imidazol-2-yl)-5,6,7,8-tetrahydro-naphthalen-2-ylamine

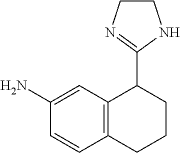

rac-8-(4,5-Dihydro-1H-imidazol-2-yl)-5,6,7,8-tetrahydro-naphthalen-2-ylamine was prepared from 7-nitro-3,4-dihydro-2H-naphthalen-1-one in analogy to Example 3 providing rac-2-(7-nitro-1,2,3,4-tetrahydro-naphthalen-1-yl)-4,5-dihydro-1H-imidazole which was reduced to the title compound by methods known in the art: colorless solid; MS (ISP): 216.4 ((M+H)⁺.).

### Example 28

### 2-(2H-Chromen-4-yl)-1H-imidazole

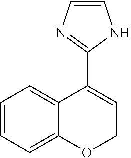

2-(2H-Chromen-4-yl)-1H-imidazole was prepared from rac-4-(1H-Imidazol-2-yl)-chroman-4-ol in analogy to Example 3b) but temperature was kept at 0° C.: light green solid: MS (ISP): 199.1 ((M+H)⁺.).

### Example 29

### 2-(6,8-Dichloro-2H-chromen-4-yl)-1H-imidazole

### a) rac-6,8-Dichloro-4-(1H-imidazol-2-yl)-chroman-4-ol

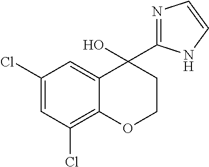

rac-6,8-Dichloro-4-(1H-imidazol-2-yl)-chroman-4-ol was prepared from 6,8-dichloro-chroman-4-one in analogy to Example 12 a): colorless solid; MS (ISP): 284.8 ((M+H)⁺.).

### b) 2-(6,8-Dichloro-2H-chromen-4-yl)-1H-imidazole

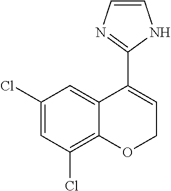

2-(6,8-Dichloro-2H-chromen-4-yl)-1H-imidazole was prepared from rac-6,8-dichloro-4-(1H-imidazol-2-yl)-chroman-4-ol in analogy to Example 3b) but temperature was kept at 0° C.: colorless solid: MS (EI): 266.1 ((M⁺.), 100%).

### Example 30

### 2-(8-Chloro-2H-chromen-4-yl)-1H-imidazole

### a) rac-8-Chloro-4-(1H-imidazol-2-yl)-chroman-4-ol

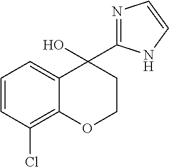

rac-8-Chloro-4-(1H-imidazol-2-yl)-chroman-4-ol was prepared from 8-chloro-chroman-4-one in analogy to Example 12 a): colorless solid; MS (ISP): 250.9 (((M+H)⁺.), 100%).

### b) 2-(8-Chloro-2H-chromen-4-yl)-1H-imidazole

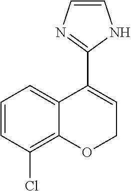

2-(8-Chloro-2H-chromen-4-yl)-1H-imidazole was prepared from rac-8-chloro-4-(1H-imidazol-2-yl)-chroman-4-ol in analogy to Example 3b) but temperature was kept at 0° C.: off-white solid: MS (EI): 232.1 ((M⁺.), 100%).

### Example 31

### 2-(6-Chloro-2H-chromen-4-yl)-1H-imidazole

### a) rac-6-Chloro-4-(1H-imidazol-2-yl)-chroman-4-ol

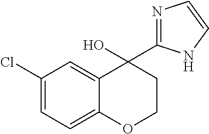

rac-6-Chloro-4-(1H-imidazol-2-yl)-chroman-4-ol was prepared from 6-chloro-chroman-4-one in analogy to Example 12 a): off-white solid; MS (ISP): 250.9 (((M+H)⁺.), 100%).

### b) 2-(6-Chloro-2H-chromen-4-yl)-1H-imidazole

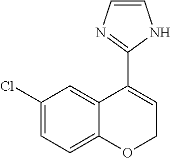

2-(6-Chloro-2H-chromen-4-yl)-1H-imidazole was prepared from rac-6-chloro-4-(1H-imidazol-2-yl)-chroman-4-ol in analogy to Example 3b) but temperature was kept at 0° C.: light brown solid: MS (EI): 232.1 ((M⁺.), 100%).

### Example 32

### rac-2-(8-Chloro-chroman-4-yl)-1H-imidazole

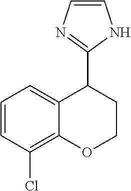

rac-2-(8-Chloro-chroman-4-yl)-1H-imidazole was prepared from 2-(8-chloro-2H-chromen-4-yl)-1H-imidazole in analogy to Example 12c): colorless solid: MS (EI): 234.2 (M⁺.), 219.1 (((M−CH₃)⁺.), 100%).

### Example 33

### rac-2-(6-Chloro-chroman-4-yl)-1H-imidazole

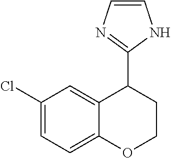

rac-2-(6-Chloro-chroman-4-yl)-1H-imidazole was prepared from 2-(6-chloro-2H-chromen-4-yl)-1H-imidazole in analogy to Example 12c): colorless solid: MS (ISP): 235.1 (((M+H)⁺.), 100%).

### Example 34

### rac-2-(6-Methoxy-chroman-4-yl)-1H-imidazole

### a) rac-4-(1H-Imidazol-2-yl)-6-methoxy-chroman-4-ol

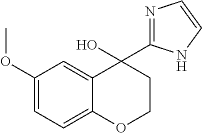

rac-4-(1H-Imidazol-2-yl)-6-methoxy-chroman-4-ol was prepared from 6-methoxy-chroman-4-one in analogy to Example 12 a): off-white solid; MS (ISP): 247.0 ((M+H)⁺.).

### b) 2-(6-Methoxy-2H-chromen-4-yl)-1H-imidazole

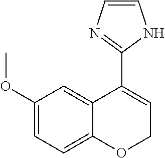

2-(6-Methoxy-2H-chromen-4-yl)-1H-imidazole was prepared from rac-6-fluoro-4-(1H-imidazol-2-yl)-chroman-4-ol in analogy to Example 12b): off-white solid: MS (EI): 228.2 ((M⁺.), 100%), 213.1 ((M−CH₃)⁺.).

### c) rac-2-(6-Methoxy-chroman-4-yl)-1H-imidazole

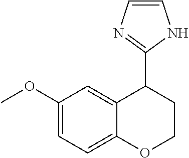

rac-2-(6-Methoxy-chroman-4-yl)-1H-imidazole was prepared from 2-(6-methoxy-2H-chromen-4-yl)-1H-imidazole in analogy to Example 12c): off-white solid: MS (EI): 230.2 ((M⁺.), 100%), 215.2 ((M−CH₃)⁺.).

### Example 35

### rac-2-(8-Methoxy-chroman-4-yl)-1H-imidazole

### a) rac-4-(1H-Imidazol-2-yl)-8-methoxy-chroman-4-ol

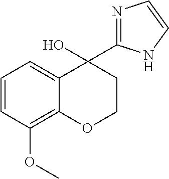

rac-4-(1H-Imidazol-2-yl)-8-methoxy-chroman-4-ol was prepared from 8-methoxy-chroman-4-one in analogy to Example 12 a): colorless solid; MS (EI): 246.2 (M⁺.), 228.2 ((M−H₂O)⁺.), 95.2 (((C(═O)-2-imidazolyl)⁺.), 100%).

### b) 2-(8-Methoxy-2H-chromen-4-yl)-1H-imidazole

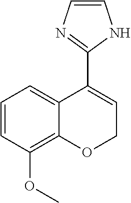

2-(8-Methoxy-2H-chromen-4-yl)-1H-imidazole was prepared from rac-4-(1H-imidazol-2-yl)-8-methoxy-chroman-4-ol in analogy to Example 12b): off-white solid: MS (EI): 228.1 ((M⁺.), 100%).

### c) rac-2-(8-Methoxy-chroman-4-yl)-1H-imidazole

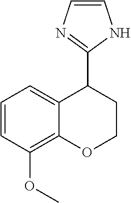

rac-2-(8-Methoxy-chroman-4-yl)-1H-imidazole was prepared from 2-(8-methoxy-2H-chromen-4-yl)-1H-imidazole in analogy to Example 12c): colorless solid: MS (ISP): 231.1 ((M+H)⁺.).

### Example 36

### rac-2-(6,8-Dichloro-chroman-4-yl)-1H-imidazole

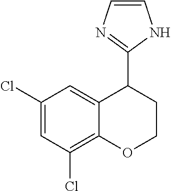

rac-2-(6,8-Dichloro-chroman-4-yl)-1H-imidazole was prepared from 2-(6,8-dichloro-2H-chromen-4-yl)-1H-imidazole in analogy to Example 3b) but temperature was kept at ambient temperature for 2 hours: colorless solid: MS (EI): 268.1 (M⁺.), 253.1 (((M−CH₃)⁺.), 100%).

### Example 37

### rac-2-(7-Methyl-chroman-4-yl)-1H-imidazole

### a) rac-4-(1H-Imidazol-2-yl)-7-methyl-chroman-4-ol

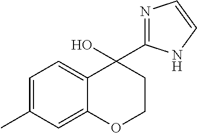

rac-4-(1H-Imidazol-2-yl)-7-methyl-chroman-4-ol was prepared from 7-methyl-chroman-4-one in analogy to Example 12 a): colorless solid; MS (EI): 230.2 (M⁺.), 212.2 ((M−H₂O)⁺.), 183.2 ((M−(H₂O+H+CO))⁺.), 95.2 (((C(═O)-2-imidazolyl)⁺.), 100%).

### b) 2-(7-Methyl-2H-chromen-4-yl)-1H-imidazole

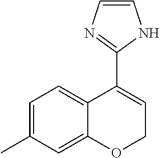

2-(7-Methyl-2H-chromen-4-yl)-1H-imidazole was prepared from rac-4-(1H-imidazol-2-yl)-7-methyl-chroman-4-ol in analogy to Example 12b): light yellow solid: MS (EI): 212.2 ((M⁺.), 100%).

### c) rac-2-(7-Methyl-chroman-4-yl)-1H-imidazole

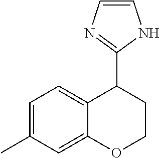

rac-2-(7-Methyl-chroman-4-yl)-1H-imidazole was prepared from 2-(7-methyl-2H-chromen-4-yl)-1H-imidazole in analogy to Example 3b) but temperature was kept at ambient temperature for 18 hours: colorless solid: MS (EI): 214.2 (M⁺.), 199.2 (((M−CH₃)⁺.), 100%).

### Example 38

### 2-(5-Methyl-2H-chromen-4-yl)-1H-imidazole

### a) rac-4-(1H-Imidazol-2-yl)-5-methyl-chroman-4-ol

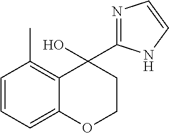

rac-4-(1H-Imidazol-2-yl)-5-methyl-chroman-4-ol was prepared from 5-methyl-chroman-4-one in analogy to Example 12 a): colorless solid; MS (EI): 230.2 ((M⁺.), 100%), 95.2 ((C(═O)-2-imidazolyl)⁺.).

### b) 2-(5-Methyl-2H-chromen-4-yl)-1H-imidazole

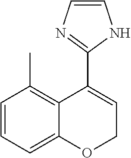

2-(5-Methyl-2H-chromen-4-yl)-1H-imidazole was prepared by heating a solution of rac-4-(1H-imidazol-2-yl)-5-methyl-chroman-4-ol in 4N aqueous HCl for 16 hours. The reaction mixture was cooled to ambient temperature, pH adjusted to 10 by addition of ammonia and extracted with tert.-butyl methyl ether. The collected organic phases were washed with brine, dried over Na₂SO₄, filtered and evaporated: colorless solid: MS (ISP): 213.1 ((M+H)⁺.).

### Example 39

### rac-2-(7-Fluoro-chroman-4-yl)-1H-imidazole

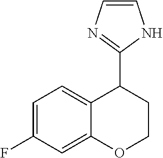

rac-2-(7-Fluoro-chroman-4-yl)-1H-imidazole was prepared from 7-fluoro-chroman-4-one in analogy to Example 13: colorless solid; MS (EI): 218.1 (M⁺.), 203.1 (((M−CH₃)⁺.), 100%).

### Example 40

### rac-4-(1H-Imidazol-2-yl)-1-(toluene-4-sulfonyl)-1,2,3,4-tetrahydro-quinoline

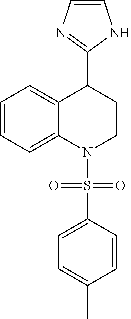

### a) 1-Phenyl-azetidin-2-one

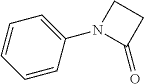

Phenyl-azetidin-2-one was prepared from azetidin-2-one and iodobenzene by treatment with trans-1,2-diaminocyclohexane, copper(I) iodide and potassium carbonate according to the procedure described in J. Am. Chem. Soc. 2001, 123, 7727-7729; off-white crystalline solid; MS (ISP): 148.4 ([M+H]⁺, 100%).

### b) 2,3-Dihydro-4(1H)-quinolinone

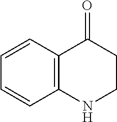

2,3-Dihydro-4(1H)-quinolinone was prepared from 1-phenyl-azetidin-2-one by treatment with trifluoromethanesulfonic acid in 1,2-dichloroethane according to the procedure described in Tetrahedron 2002, 58, 8475-8481; yellow oil; MS (ISP): 148.3 ([M+H]⁺, 100%).

### c) 1-(Toluene-4-sulfonyl)-2,3-dihydro-1H-quinolin-4-one

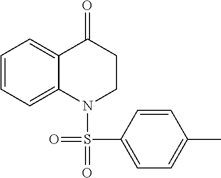

To a solution of 2.57 g (17.5 mmol) 2,3-dihydro-4(1H)-quinolinone in 20 ml dichloromethane at 0° C. was added dropwise 9.09 ml (65.6 mmol) triethylamine. Then 5.25 g (27.5 mmol) p-toluenesulphonyl chloride was added and the reaction mixture was heated at reflux for 16 hours. After cooling to room temperature the mixture was diluted with dichloromethane and washed sequentially with 1 M aqueous hydrochloric acid, saturated aqueous sodium bicarbonate, and saturated brine. The phases were separated and the organic phase was dried over Na₂SO₄, filtered and concentrated in vacuo. The residue was purified by chromatography (silica gel, ethyl acetate/heptane) to afford 1.55 g (29%) of the title compound as a white crystalline solid. MS (ISP): 302.4 ([M+H]⁺, 100%).

### d) rac-4-(1H-Imidazol-2-yl)-1-(toluene-4-sulfonyl)-1,2,3,4-tetrahydro-quinolin-4-ol

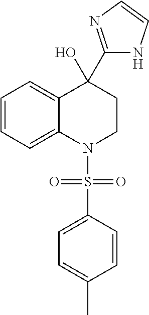

rac-4-(1H-Imidazol-2-yl)-1-(toluene-4-sulfonyl)-1,2,3,4-tetrahydro-quinolin-4-ol was prepared from 1-(toluene-4-sulfonyl)-2,3-dihydro-1H-quinolin-4-one and 2-(1-diethoxymethyl-1H-imidazol-2-yl)-lithium in analogy to Example 12 a): off-white foam; MS (ISP): 370.1 ([M+H]⁺, 100%).

### e) 4-(1H-Imidazol-2-yl)-1-(toluene-4-sulfonyl)-1,2-dihydro-quinoline

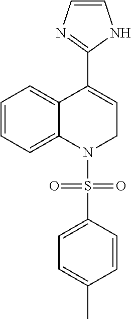

4-(1H-Imidazol-2-yl)-1-(toluene-4-sulfonyl)-1,2-dihydro-quinoline was prepared from rac-4-(1H-imidazol-2-yl)-1-(toluene-4-sulfonyl)-1,2,3,4-tetrahydro-quinolin-4-ol and sulfuric acid in ethanol at 0-20° C. in analogy to Example 12b): white crystalline solid: MS (ISP): 352.3 ([M+H]⁺, 100%).

### f) rac-4-(1H-Imidazol-2-yl)-1-(toluene-4-sulfonyl)-1,2,3,4-tetrahydro-quinoline

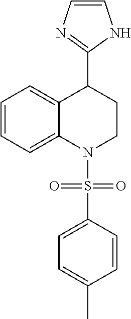

rac-4-(1H-Imidazol-2-yl)-1-(toluene-4-sulfonyl)-1,2,3,4-tetrahydro-quinoline was prepared from 4-(1H-imidazol-2-yl)-1-(toluene-4-sulfonyl)-1,2-dihydro-quinoline and lithium aluminium hydride in tetrahydrofuran at reflux in analogy to Example 12c): off-white foam; MS (ISP): 354.3 ([M+H]⁺, 100%)

### Example 41

### rac-4-(1H-Imidazol-2-yl)-1,2,3,4-tetrahydro-quinoline

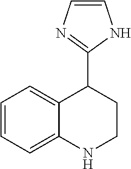

rac-4-(1H-Imidazol-2-yl)-1,2,3,4-tetrahydro-quinoline was obtained as a by-product during the preparation of rac-4-(1H-imidazol-2-yl)-1-(toluene-4-sulfonyl)-1,2,3,4-tetrahydro-quinoline as described in Example 40f): off-white amorphous solid; MS (ISP): 200.4 ([M+H]⁺, 100%).

### Example 42

### rac-2-(5-Methyl-chroman-4-yl)-1H-imidazole

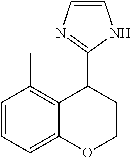

rac-2-(5-Methyl-chroman-4-yl)-1H-imidazole was prepared from 2-(5-methyl-2H-chromen-4-yl)-1H-imidazole by hydrogenation at 100 bar with 10% Pd/C as catalyst in ethyl acetate at 50° C. for 18 hours. After usual workup the residue was purified by flash-chromatography on silica gel with a gradient of ethyl acetate/methanol 5%-30% as eluent: colorless solid; MS (ISP): 215.2 ((M+H)⁺.).

### Example 43

### rac-2-(5-Fluoro-chroman-4-yl)-1H-imidazole

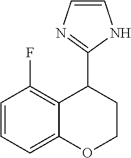

rac-2-(5-Fluoro-chroman-4-yl)-1H-imidazole was prepared from 5-fluoro-chroman-4-one in analogy to Example 13: colorless solid; MS (ISP): 219.1 ((M+H)⁺.).

### Example 44

### rac-4-(1H-Imidazol-2-yl)-1-methyl-1,2,3,4-tetrahydro-quinoline

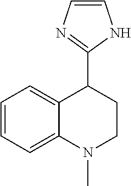

### a) 1-Methyl-2,3-dihydro-1H-quinolin-4-one

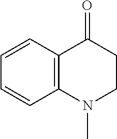

This compound was prepared according to the procedure described in J. Med. Chem. 2003, 46, 1962-1979. To a solution of 220 mg (1.49 mmol) 2,3-dihydro-4(1H)-quinolinone in 3 ml acetone in a pressure tube was added 620 mg (4.48 mmol) potassium carbonate. Then 0.38 ml (5.98 mmol) iodomethane was added dropwise, the tube was sealed, and the reaction mixture was heated at 80° C. for 16 hours. After cooling to room temperature the mixture was diluted with ethyl acetate and washed with saturated brine. The phases were separated and the organic phase was dried over Na₂SO₄, filtered and concentrated in vacuo. The residue was purified by chromatography (silica gel, ethyl acetate/heptane) to afford 147 mg (61%) of the title compound as a light yellow oil. MS (ISP): 162.1 ([M+H]⁺, 100%).

### b) rac-4-(1H-Imidazol-2-yl)-1-methyl-1,2,3,4-tetrahydro-quinolin-4-ol

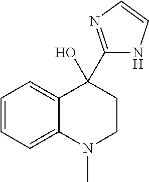

rac-4-(1H-Imidazol-2-yl)-1-methyl-1,2,3,4-tetrahydro-quinolin-4-ol was prepared from 1-methyl-2,3-dihydro-1H-quinolin-4-one and 2-(1-diethoxymethyl-1H-imidazol-2-yl)-lithium in analogy to Example 12 a): off-white crystalline solid; MS (ISP): 230.4 ([M+H]⁺, 54%), 212.1 ([M+H—H₂O]⁺, 100%).

### c) 4-(1H-Imidazol-2-yl)-1-methyl-1,2-dihydro-quinoline

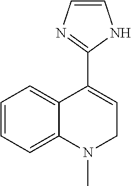

4-(1H-Imidazol-2-yl)-1-methyl-1,2-dihydro-quinoline was prepared from rac-4-(1H-imidazol-2-yl)-1-methyl-1,2,3,4-tetrahydro-quinolin-4-ol and sulfuric acid in ethanol at 50° C. in analogy to Example 12b): orange crystalline solid: MS (ISP): 212.3 ([M+H]⁺, 100%).

### d) rac-4-(1H-Imidazol-2-yl)-1-methyl-1,2,3,4-tetrahydro-quinoline

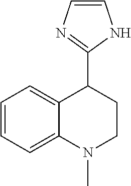

rac-4-(1H-Imidazol-2-yl)-1-methyl-1,2,3,4-tetrahydro-quinoline was prepared from 4-(1H-imidazol-2-yl)-1-methyl-1,2-dihydro-quinoline and lithium aluminium hydride in tetrahydrofuran at reflux in analogy to Example 12c): off-white crystalline solid; MS (ISP): 214.1 ([M+H]⁺, 100%).

### Example 45

### 2-(3-Methyl-2H-chromen-4-yl)-1H-imidazole

### a) rac-4-(1H-Imidazol-2-yl)-3-methyl-chroman-4-ol

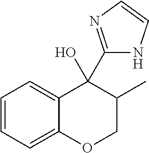

rac-4-(1H-Imidazol-2-yl)-3-methyl-chroman-4-ol was prepared from 3-methyl-chroman-4-one in analogy to Example 12 a): colorless solid; MS (ISP): 231.1 ((M+H)⁺.).

### b) 2-(3-Methyl-2H-chromen-4-yl)-1H-imidazole

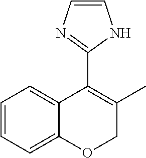

2-(3-Methyl-2H-chromen-4-yl)-1H-imidazole was prepared from rac-4-(1H-imidazol-2-yl)-3-methyl-chroman-4-ol in analogy to Example 38b): light brown solid; MS (ISP): 213.0 ((M+H)⁺.).

### Example 46

### 2-(2,2-Dimethyl-2H-chromen-4-yl)-1H-imidazole

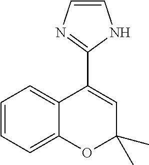

2-(2,2-Dimethyl-2H-chromen-4-yl)-1H-imidazole was prepared from 2,2-dimethyl-chroman-4-one in analogy to Example 38: yellow solid; MS (ISP): 227.0 ((M+H)⁺.).

### Example 47

### rac-2-(2,2-Dimethyl-chroman-4-yl)-1H-imidazole

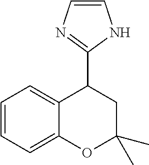

rac-2-(2,2-Dimethyl-chroman-4-yl)-1H-imidazole was prepared from 2-(2,2-dimethyl-2H-chromen-4-yl)-1H-imidazole in analogy to Example 42: colorless solid; MS (ISP): 229.2 ((M+H)⁺.).

### Example 48

### rac-2-(2-Methyl-2H-chromen-4-yl)-1H-imidazole

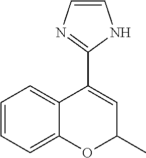

rac-2-(2-Methyl-2H-chromen-4-yl)-1H-imidazole was prepared from rac-2-methyl-chroman-4-one in analogy to Example 38: light brown solid; MS (ISP): 213.0 ((M+H)⁺.).

### Example 49

### (3R,4S or 3S,4R)-2-(3-Methyl-chroman-4-yl)-1H-imidazole

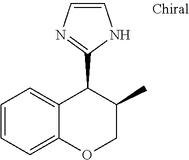

(3R,4S or 3S,4R)-2-(3-Methyl-chroman-4-yl)-1H-imidazole was obtained by chromatographic separation of a diastereomeric mixture of rac-2-(3-methyl-chroman-4-yl)-1H-imidazole (Example 53) on a Chiralpak AD column with heptane/ethanol 93:7 as eluent: colorless solid; MS (ISP): 215.1 ((M+H)⁺.).

### Example 50

### rac-2-(2-Methyl-chroman-4-yl)-1H-imidazole

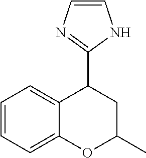

rac-2-(2-Methyl-chroman-4-yl)-1H-imidazole was prepared from rac-2-(2-methyl-2H-chromen-4-yl)-1H-imidazole in analogy to Example 42: light green solid; MS (ISP): 215.1 ((M+H)⁺.).

### Example 51

### (2R,4S or 2S,4R)-2-(2-Methyl-chroman-4-yl)-1H-imidazole

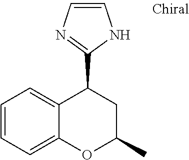

(2R,4S or 2S,4R)-2-(2-Methyl-chroman-4-yl)-1H-imidazole was obtained from rac-2-(2-methyl-chroman-4-yl)-1H-imidazole in analogy to Example 49: colorless solid; MS (ISP): 215.1 ((M+H)⁺.).

### Example 52

### (2S,4R or 2R,4S)-2-(2-Methyl-chroman-4-yl)-1H-imidazole

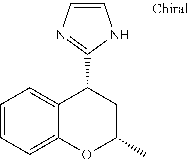

(2S,4R or 2R,4S)-2-(2-Methyl-chroman-4-yl)-1H-imidazole was obtained from rac-2-(2-methyl-chroman-4-yl)-1H-imidazole in analogy to Example 49: colorless solid; MS (ISP): 215.1 ((M+H)⁺.).

### Example 53

### rac-2-(3-Methyl-chroman-4-yl)-1H-imidazole

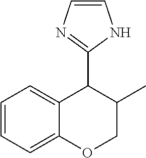

rac-2-(3-Methyl-chroman-4-yl)-1H-imidazole was prepared from rac-4-(1H-imidazol-2-yl)-3-methyl-chroman-4-ol by reduction with lithium in liquid ammonia for 30 min. The blue reaction mixture was quenched by addition of solid ammonium chloride, the ammonia evaporated and the residue distributed between water and t-butyl methyl ether. The organic phase was washed with brine, dried over sodium sulfate, filtered and evaporated. rac-2-(3-Methyl-chroman-4-yl)-1H-imidazole was obtained as colorless solid; MS (ISP): 215.1 ((M+H)⁺.).

### Example 54

### (3S,4S and 3R,4R)-2-(3-Methyl-chroman-4-yl)-1H-imidazole

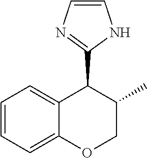

The racemic mixture of (3S,4S and 3R,4R)-2-(3-methyl-chroman-4-yl)-1H-imidazole was obtained (together with both separated cis-isomers) from rac-2-(3-methyl-chroman-4-yl)-1H-imidazole in analogy to Example 49: colorless solid; MS (ISP): 215.1 ((M+H)⁺.).

### Example 55

### (4-(3,4-Dihydro-naphthalen-1-yl)-1H-imidazole

### a) 1-(1-Trityl-1H-imidazol-4-yl)-1,2,3,4-tetrahydro-naphthalen-1-ol

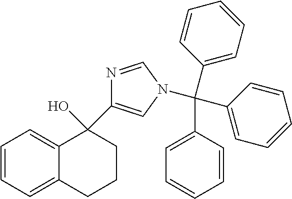

1-(1-Trityl-1H-imidazol-4-yl)-1,2,3,4-tetrahydro-naphthalen-1-ol was prepared from 4-iodo-1-trityl-1H-imidazole and alpha-tetralone following the procedure described by X. Zhang et al., J. Med. Chem. 40, 3014 (1997): colorless solid; MS (ISP): 457.5 ((M+H)⁺.).

### b) (4-(3,4-Dihydro-naphthalen-1-yl)-1H-imidazole

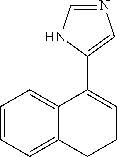

4-(3,4-Dihydro-naphthalen-1-yl)-1H-imidazole was prepared by reaction of 1-(1-trityl-1H-imidazol-4-yl)-1,2,3,4-tetrahydro-naphthalen-1-ol with a solution of trifluoroacetic acid in water 6:4 following the procedure described by X. Zhang et al., J. Med. Chem. 40, 3014 (1997): colorless solid; MS (ISP): 197.3 ((M+H)⁺.).

### Example 56

### rac-4-(1,2,3,4-Tetrahydro-naphthalen-1-yl)-1H-imidazole

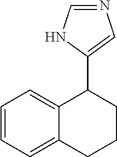

rac-4-(1,2,3,4-Tetrahydro-naphthalen-1-yl)-1H-imidazole was prepared from 4-(3,4-dihydro-naphthalen-1-yl)-1H-imidazole in analogy to Example 42 but hydrogen pressure was kept at 3.5 bar and the reaction run at ambient temperature for 5 hours: colorless solid; MS (EI): 198.2 ((M⁺.), 100%).

### Example 57

### (4-(3,4-Dihydro-naphthalen-1-yl)-1H-imidazole

### a) 2-(tert-Butyl-dimethyl-silanyl)-4-(1-hydroxy-1,2,3,4-tetrahydro-naphthalen-1-yl)-imidazole-1-sulfonic acid dimethylamide

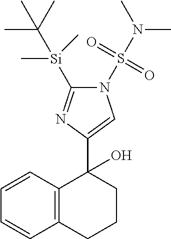

2-(tert-Butyl-dimethyl-silanyl)-4-(1-hydroxy-1,2,3,4-tetrahydro-naphthalen-1-yl)-imidazole-1-sulfonic acid dimethylamide was prepared from 2-(tert-butyl-dimethyl-silanyl)-imidazole-1-sulfonic acid dimethylamide and chroman-4-one in analogy to the procedure published by S. Ohta et al., Synthesis 1990, 78: light brown viscous oil; MS (ISP): 438.5 ((M+H)⁺.).

### b) 5-(2H-Chromen-4-yl)-1H-imidazole or tautomer

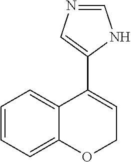

5-(2H-Chromen-4-yl)-1H-imidazole was prepared from 2-(tert-butyl-dimethyl-silanyl)-4-(1-hydroxy-1,2,3,4-tetrahydro-naphthalen-1-yl)-imidazole-1-sulfonic acid dimethylamide in analogy to Example 38b) but in aqueous 2N HCl solution at reflux for 2 hours: colorless solid; MS (EI): 198.2 ((M⁺.), 100%).

### Example 58

### 5-(6-Fluoro-2H-chromen-4-yl)-1H-imidazole or tautomer

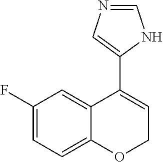

5-(6-Fluoro-2H-chromen-4-yl)-1H-imidazole was prepared from 2-(tert-butyl-dimethyl-silanyl)-imidazole-1-sulfonic acid dimethylamide and 6-fluoro-chroman-4-one in analogy to Example 57: off-white solid; MS (EI): 216.1 ((M⁺.), 100%).

### Example 59

### 5-(7-Methyl-2H-chromen-4-yl)-1H-imidazole or tautomer

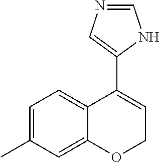

5-(7-Methyl-2H-chromen-4-yl)-1H-imidazole was prepared from 2-(tert-butyl-dimethyl-silanyl)-imidazole-1-sulfonic acid dimethylamide and 7-methyl-chroman-4-one in analogy to Example 57: light brown solid; MS (EI): 212.2 ((M⁺.), 100%).

### Example 60

### rac-5-(7-Methyl-chroman-4-yl)-1H-imidazole or tautomer

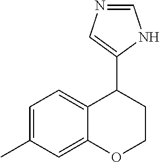

rac-5-(7-Methyl-chroman-4-yl)-1H-imidazole was prepared from 5-(7-methyl-2H-chromen-4-yl)-1H-imidazole in analogy to Example 42: colorless solid; MS (EI): 214.2 ((M⁺.), 100%).

### Example 61

### 5-(5-Fluoro-2H-chromen-4-yl)-1H-imidazole or tautomer

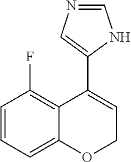

5-(5-Fluoro-2H-chromen-4-yl)-1H-imidazole was prepared from 2-(tert-butyl-dimethyl-silanyl)-imidazole-1-sulfonic acid dimethylamide and 5-fluoro-chroman-4-one in analogy to Example 57: colorless solid; MS (EI): 216.2 ((M⁺.), 100%).

### Example 62

### rac-5-(6-Fluoro-chroman-4-yl)-1H-imidazole or tautomer

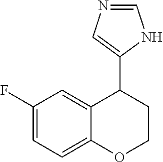

rac-5-(6-Fluoro-chroman-4-yl)-1H-imidazole was prepared from 5-(6-fluoro-2H-chromen-4-yl)-1H-imidazole in analogy to Example 12c): colorless solid; MS (EI): 218.2 ((M⁺.), 100%).

### Example 63

### 5-(8-Chloro-2H-chromen-4-yl)-1H-imidazole or tautomer

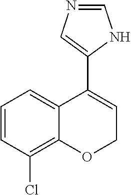

5-(8-Chloro-2H-chromen-4-yl)-1H-imidazole was prepared from 2-(tert-butyl-dimethyl-silanyl)-imidazole-1-sulfonic acid dimethylamide and 8-chloro-chroman-4-one in analogy to Example 57: off-white solid; MS (EI): 232.1 ((M⁺.), 100%).

### Example 64

### 5-(6-Chloro-2H-chromen-4-yl)-1H-imidazole or tautomer

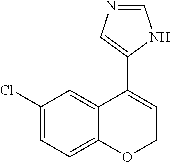

5-(6-Chloro-2H-chromen-4-yl)-1H-imidazole was prepared from 2-(tert-butyl-dimethyl-silanyl)-imidazole-1-sulfonic acid dimethylamide and 6-chloro-chroman-4-one in analogy to Example 57: off-white solid; MS (EI): 232.1 ((M⁺.), 100%).

### Example 65

### rac-5-(7-Fluoro-chroman-4-yl)-1H-imidazole or tautomer

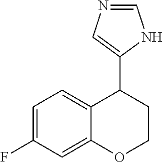

rac-5-(7-Fluoro-chroman-4-yl)-1H-imidazole was prepared from 5-(7-fluoro-2H-chromen-4-yl)-1H-imidazole in analogy to Example 42: colorless solid; MS (ISP): 219.1 ((M+H)⁺.).

### Example 66

### rac-5-(5-Methyl-chroman-4-yl)-1H-imidazole or tautomer

### a) 5-(5-Methyl-2H-chromen-4-yl)-1H-imidazole or tautomer

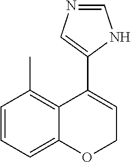

5-(5-Methyl-2H-chromen-4-yl)-1H-imidazole was prepared from 2-(tert-butyl-dimethyl-silanyl)-imidazole-1-sulfonic acid dimethylamide and 5-methyl-chroman-4-one in analogy to Example 57: colorless solid; MS (EI): 212.2 ((M⁺.), 100%).

### b) rac-5-(5-Methyl-chroman-4-yl)-1H-imidazole or tautomer

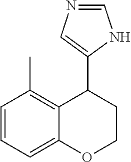

rac-5-(5-Methyl-chroman-4-yl)-1H-imidazole was prepared from 5-(5-methyl-2H-chromen-4-yl)-1H-imidazole in analogy to Example 42: colorless solid; MS (ISP): 215.2 ((M+H)⁺.).

### Example 67

### rac-5-(5-Fluoro-chroman-4-yl)-1H-imidazole or tautomer

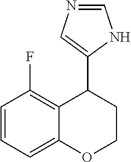

rac-5-(5-Fluoro-chroman-4-yl)-1H-imidazole was prepared from 5-(5-fluoro-2H-chromen-4-yl)-1H-imidazole in analogy to Example 42: colorless solid; MS (ISP): 219.1 ((M+H)⁺.).

### Example 68

### 5-(7-Fluoro-2H-chromen-4-yl)-1H-imidazole or tautomer

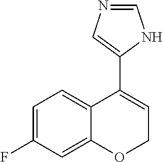

5-(7-Fluoro-2H-chromen-4-yl)-1H-imidazole was prepared from 2-(tert-butyl-dimethyl-silanyl)-imidazole-1-sulfonic acid dimethylamide and 7-fluoro-chroman-4-one in analogy to Example 57: light brown solid; MS (ISP): 217.1 ((M+H)⁺.).

### Example 69

### rac-5-Chroman-4-yl-1H-imidazole hydrochloride or tautomer

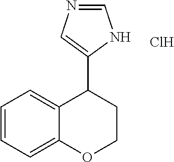

rac-5-Chroman-4-yl-1H-imidazole was prepared from 5-(2H-chromen-4-yl)-1H-imidazole in analogy to Example 12c). Compound was isolated as hydrochloride: off-white solid; MS (ISP): 201.1 ((M+H)⁺.).

### Example 70

### 5-(3-Methyl-2H-chromen-4-yl)-1H-imidazole or tautomer

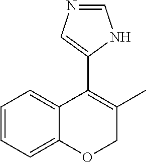

5-(3-Methyl-2H-chromen-4-yl)-1H-imidazole was prepared from 2-(tert-butyl-dimethyl-silanyl)-imidazole-1-sulfonic acid dimethylamide and 3-methyl-chroman-4-one in analogy to Example 57: light brown solid; MS (ISP): 213.0 ((M+H)⁺.).

### Example 71

### rac-1-(1H-Imidazol-4-yl)-1,2,3,4-tetrahydro-naphthalen-1-ol or tautomer

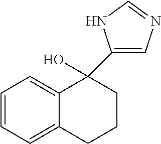

rac-1-(1H-Imidazol-4-yl)-1,2,3,4-tetrahydro-naphthalen-1-ol was prepared from rac-1-(1-trityl-1H-imidazol-4-yl)-1,2,3,4-tetrahydro-naphthalen-1-ol (Example 55 a)) by deprotection with formic acid/tetrahydrofuran/water 1:1:0.1 in analogy of a procedure described by A. Ojima et al., Org. Lett. 4, 3051 (2002): colorless solid; MS (ISP): 215.3 ((M+H)⁺.).

### Example 72

### rac-2-Chroman-4-ylmethyl-1H-imidazole

### a) rac-Chroman-4-carboxylic acid methoxy-methyl-amide

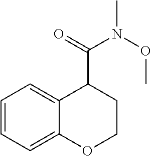

To a solution of 500 mg (2.8 mmol) chroman-4-carboxylic acid in 10 ml dichloromethane were added 330 mg (3.2 mmol) N,O-dimethylhydroxylamine hydrochloride and 993 mg (3.2 mmol) N-(3-dimethylaminopropyl)-N′-ethyl-carbodiimide hydrochloride and the mixture stirred at ambient temperature for 5 min. Then 710 mg (0.98 ml, 10 mmol) triethylamine were added drop-wise and the resulting mixture stirred at ambient temperature for 4 hours. For workup 2M HCl solution was added, the organic solvent evaporated and the residue extracted with tert-butyl methyl ether. the combined organic phase was washed with brine, dried over Na2SO4, filtered and evaporated: 503 mg rac-chroman-4-carboxylic acid methoxy-methyl-amide as light brown oil; MS (EI): 221.2 (M⁺.), 133.1 (((M−C(═O)N(CH₃)OCH₃)⁺.), 100%).

### b) rac-Chroman-4-yl-(1H-imidazol-2-yl)-methanone

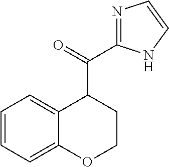

rac-Chroman-4-yl-(1H-imidazol-2-yl)-methanone was prepared from rac-chroman-4-carboxylic acid methoxy-methyl-amide in analogy to Example 12 a): colorless gum; MS (ISP): 228.9 ((M+H)⁺.).

### c) rac-2-Chroman-4-ylmethyl-1H-imidazole

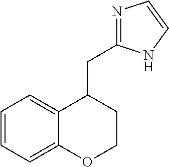

rac-2-Chroman-4-ylmethyl-1H-imidazole was prepared from rac-chroman-4-yl-(1H-imidazol-2-yl)-methanone in a Wolff-Kishner type reduction following the published procedure of E. Reimann et al., Arch. Pharm. (Weinheim) 322, 363 (1989): yellow gum; MS (ISP): 215.1 M+H)⁺.).

### Example 73

### rac-4-(1H-Imidazol-2-ylmethyl)-1,2,3,4-tetrahydro-quinoline

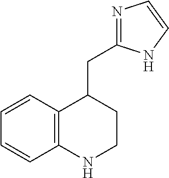

### a) rac-1,2,3,4-Tetrahydro-quinoline-4-carboxylic acid

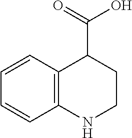

rac-1,2,3,4-Tetrahydro-quinoline-4-carboxylic acid was prepared from quinoline-4-carboxylic acid by treatment with Raney nickel in aqueous sodium hydroxide according to the procedure described in Khimiya Geterotsiklicheskikh Soedinenii 1988, 77-9; brown crystals; ¹H-NMR (CDCl₃): 2.04 (1H, m), 2.29 (1H, m), 3.24-3.46 (br m, 3H, CH₂N and NH), 3.77 (1H, t, CHCO₂), 6.55 (1H, d, ArH), 6.67 (1H, dd, ArH), 7.04 (1H, dd, ArH), 7.15 (1H, d, ArH).

### b) rac-1,2,3,4-Tetrahydro-quinoline-4-carboxylic acid methoxy-methyl-amide

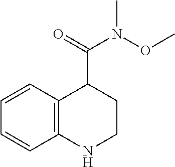

To a solution of 0.50 g (2.82 mmol) rac-1,2,3,4-tetrahydro-quinoline-4-carboxylic acid in 12 ml dichloromethane were added 0.36 g (3.67 mmol) N,O-dimethylhydroxylamine hydrochloride and 0.40 ml (3.67 mmol) N-methylmorpholine. The mixture was cooled to 0° C., then 0.70 g (3.67 mmol) 1-ethyl-3-(3′-dimethylaminopropyl)carbodiimide hydrochloride (EDCI) was added and the reaction mixture was stirred at room temperature for 16 hours. The mixture was then concentrated in vacuo and the residue was purified by chromatography (silica gel, ethyl acetate/heptane gradient) to afford 0.29 g (47%) of the title compound as a yellow crystalline solid. MS (ISP): 221.4 ([M+H]⁺, 100%).

### c) rac-1-(Toluene-4-sulfonyl)-1,2,3,4-tetrahydro-quinoline-4-carboxylic acid methoxy-methyl-amide

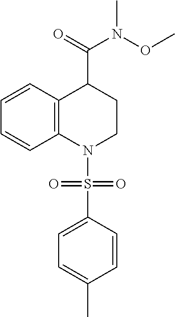

To a solution of 0.29 g (1.32 mmol) rac-1,2,3,4-tetrahydro-quinoline-4-carboxylic acid methoxy-methyl-amide in 5 ml 1,2-dichloroethane was added dropwise 0.46 ml (3.29 mmol) triethylamine. Then 0.33 g (1.71 mmol) p-toluenesulphonyl chloride was added and the reaction mixture was heated at 70° C. for 4 hours. After cooling to room temperature the mixture was concentrated in vacuo and the residue was purified by chromatography (silica gel, methanol/dichloromethane gradient) to afford 0.44 g (90%) of the title compound as a brown crystalline solid. MS (ISP): 375.1 ([M+H]⁺, 100%).

### d) rac-(1H-Imidazol-2-yl)-[1-(toluene-4-sulfonyl)-1,2,3,4-tetrahydro-quinolin-4-yl]-methanone

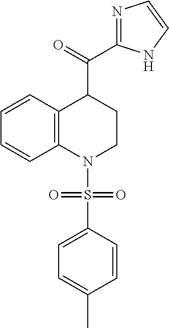

To a solution of 0.21 ml (1.29 mmol) 1-(diethoxymethyl)imidazole in 2 ml tetrahydrofuran at −78° C. was added dropwise 0.88 ml (1.41 mmol) of a 1.6 M solution of n-butyllithium in hexane. The resulting solution of 2-(1-diethoxymethyl-1H-imidazol-2-yl)-lithium was stirred at −78° C., and then added dropwise to a solution of 0.44 g (1.18 mmol) rac-1-(toluene-4-sulfonyl)-1,2,3,4-tetrahydro-quinoline-4-carboxylic acid methoxy-methyl-amide in 4 ml tetrahydrofuran at 0° C. The reaction mixture was then stirred at 0° C. for 1 h, before being quenched by dropwise addition of 2 M aqueous hydrochloric acid. The mixture was made basic by addition of aqueous sodium bicarbonate solution and diluted with ethyl acetate. The phases were separated and the organic phase was washed with saturated brine, dried over Na₂SO₄, filtered and concentrated in vacuo. The residue was purified by chromatography (silica gel, ethyl acetate/heptane gradient) to afford 0.23 g (52%) of the title compound as a light yellow crystalline solid. MS (ISP): 382.3 ([M+H]⁺, 100%).

### e) rac-4-(1H-Imidazol-2-ylmethyl)-1-(toluene-4-sulfonyl)-1,2,3,4-tetrahydro-quinoline

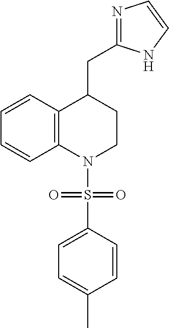

This compound was prepared following methodology reported in Arch. Pharm. 1989, 322, 363-367. To a solution of 0.23 g (0.60 mmol) rac-(1H-imidazol-2-yl)-[1-(toluene-4-sulfonyl)-1,2,3,4-tetrahydro-quinolin-4-yl]-methanone in 3 ml triethylene glycol were added sequentially 96 mg (2.41 mmol) sodium hydroxide powder and 0.10 ml (1.99 mmol) hydrazine hydrate. The reaction mixture was stirred at 110° C. for 1 h, and then at 200° C. for 2 h. After cooling to room temperature the mixture was diluted with ethyl acetate and washed sequentially with 2 N aqueous hydrochloric acid, aqueous sodium bicarbonate solution, water, and saturated brine. The phases were separated and the organic phase was dried over Na₂SO₄, filtered and concentrated in vacuo. The residue was purified by chromatography (silica gel, methanol/dichloromethane gradient) to afford 41 mg (19%) of the title compound as a yellow crystalline solid. MS (ISP): 368.1 ([M+H]⁺, 100%).

### f) rac-4-(1H-Imidazol-2-ylmethyl)-1,2,3,4-tetrahydro-quinoline

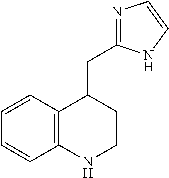

This compound was prepared following methodology reported in J. Med. Chem. 1997, 40, 105-111. To 40 mg (0.11 mmol) rac-4-(1H-imidazol-2-ylmethyl)-1-(toluene-4-sulfonyl)-1,2,3,4-tetrahydro-quinoline were added sequentially 0.57 ml (3.27 mmol) of a 33% solution of HBr in acetic acid and 0.06 ml (0.54 mmol) anisole. The reaction mixture was stirred at room temperature for 3 h and then poured onto 2 N aqueous sodium hydroxide solution. The mixture was diluted with ethyl acetate, the phases were separated, and the organic phase was washed with saturated brine. The phases were separated and the organic phase was dried over Na₂SO₄, filtered and concentrated in vacuo. The residue was purified by chromatography (silica gel, methanol/dichloromethane gradient) to afford 16 mg (69%) of the title compound as a white foam. MS (ISP): 214.3 ([M+H]⁺, 100%).

### Example 74

### rac-2-(1,2,3,4-Tetrahydro-naphthalen-1-ylmethyl)-1H-imidazole

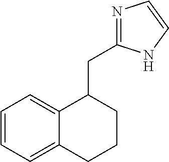

rac-2-(1,2,3,4-Tetrahydro-naphthalen-1-ylmethyl)-1H-imidazole was prepared from rac-1,2,3,4-tetrahydro-naphthalene-1-carboxylic acid methoxy-methyl-amide in analogy to Example 72b) and c): colorless solid; MS (ISP): 213.0 M+H)⁺.).

1. A method for treating a disorder selected from the group consisting of depression, anxiety disorders, bipolar disorder, attention deficit hyperactivity disorder, stress-related disorders, psychotic disorders, schizophrenia, neurological diseases, Parkinson's disease, neurodegenerative disorders, Alzheimer's disease, epilepsy, migraine, hypertension, substance abuse and metabolic disorders, eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders comprising administering to an individual a therapeutically effective amount of a compound of formula I 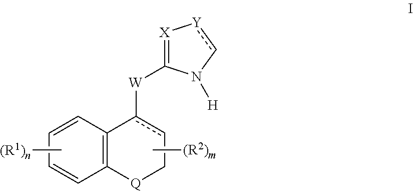
wherein
R¹ is hydrogen, tritium, hydroxy, lower alkyl, lower alkoxy, halogen, nitro, amino or lower alkyl substituted by halogen;
R² is hydrogen, hydroxy or lower alkyl;
X is N and Y is CH or CH₂ or CH-lower alkyl or
X is CH and Y is N;
Q is CH₂, O, NH, N-alkyl, N—SO₂-alkyl or N—SO₂-toluen-4-yl;
W is CH₂ or a bond
m and n are each independently 1, 2 or 3; when m is 2 or 3, R² may be the same or different; when n is 2 or 3, R¹ may be the same or different;
the dotted lines each independently represent an optional bond;
or a pharmaceutically active salt, racemic mixture, enantiomer, optical isomer or tautomeric form thereof.

2. The method of claim 1, wherein the compound is a compound of formula IA 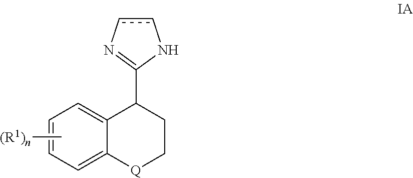
wherein
R¹ is hydrogen, tritium, hydroxy, lower alkyl, lower alkoxy, halogen or lower alkyl substituted by halogen;
Q is CH₂ or O;
n is 1, 2 or 3; when n is 2 or 3, R¹ may be the same or different;
the dotted line represents an optional bond;
or a pharmaceutically active salt, racemic mixture, enantiomer, optical isomer or tautomeric form thereof.

3. A compound of formula I 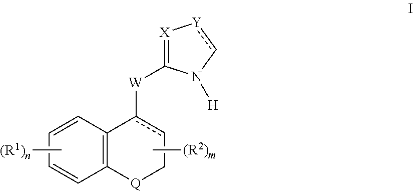
wherein
R¹ is hydrogen, tritium, hydroxy, lower alkyl, lower alkoxy, halogen, nitro, amino or lower alkyl substituted by halogen;
R² is hydrogen, hydroxy or lower alkyl;
X is N and Y is CH or CH₂ or CH-lower alkyl or
X is CH and Y is N;
Q is CH₂, O, NH, N-alkyl or N—SO₂-alkyl or N—SO₂-toluen-yl;
W is CH₂ or a bond
m and n are each independently 1, 2 or 3; when m is 2 or 3, R² may be the same or different; when n is 2 or 3, R¹ may be the same or different;
the dotted lines each independently represent an optional bond;
or a pharmaceutically active salt, racemic mixture, enantiomer, optical isomer or tautomeric form thereof, with the exception of the following compounds
rac-2-(1,2,3,4-tetrahydro-1-naphthyl)-2-imidazoline;
rac-2-(7-methyl-1,2,3,4-tetrahydro-naphthalen-1-yl)-4,5-dihydro-1H-imidazole;
rac-2-(6-methyl-1,2,3,4-tetrahydro-naphthalen-1-yl)-4,5-dihydro-1H-imidazole;
rac-2-(6-chloro-1,2,3,4-tetrahydro-naphthalen-1-yl)-4,5-dihydro-1H-imidazole;
rac-2-(5-chloro-1,2,3,4-tetrahydro-naphthalen-1-yl)-4,5-dihydro-1H-imidazole;
rac-2-(7-methoxy-1,2,3,4-tetrahydro-naphthalen-1-yl)-4,5-dihydro-1H-imidazole;
rac-2-(6-methoxy-1,2,3,4-tetrahydro-naphthalen-1-yl)-4,5-dihydro-1H-imidazole;
rac-2-(5-methoxy-1,2,3,4-tetrahydro-naphthalen-1-yl)-4,5-dihydro-1H-imidazole;
rac-5-(4,5-dihydro-1H-imidazol-2-yl)-5,6,7,8-tetrahydro-naphthalen-2-ol;
rac-4-(1,2,3,4-tetrahydro-naphthalen-1-yl)-1H-imidazole;
rac-5-(4,5-dihydro-1H-imidazol-2-yl)-5,6,7,8-tetrahydro-naphthalene-2,3-diol; and
rac-5-(4,5-dihydro-1H-imidazol-2-yl)-5,6,7,8-tetrahydro-naphthalene-1,2-diol.

4. A pharmaceutical composition comprising a therapeutically effective amount of a compound of formula I 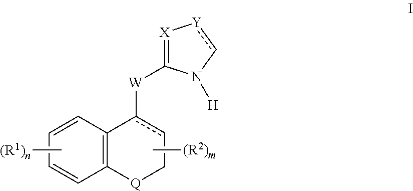
wherein
R¹ is hydrogen, tritium, hydroxy, lower alkyl, lower alkoxy, halogen, nitro, amino or lower alkyl substituted by halogen;
R² is hydrogen, hydroxy or lower alkyl;
X is N and Y is CH or CH₂ or CH-lower alkyl or
X is CH and Y is N;
Q is CH₂, O, NH, N-alkyl or N—SO₂-alkyl or N—SO₂-toluen-yl;
W is CH₂ or a bond
m and n are each independently 1, 2 or 3; when m is 2 or 3, R² may be the same or different; when n is 2 or 3, R¹ may be the same or different;
the dotted lines each independently represent an optional bond;
or a pharmaceutically active salt, racemic mixture, enantiomer, optical isomer or tautomeric form thereof, with the exception of the following compounds
rac-2-(1,2,3,4-tetrahydro-1-naphthyl)-2-imidazoline;
rac-2-(7-methyl-1,2,3,4-tetrahydro-naphthalen-1-yl)-4,5-dihydro-1H-imidazole;
rac-2-(6-methyl-1,2,3,4-tetrahydro-naphthalen-1-yl)-4,5-dihydro-1H-imidazole;
rac-2-(6-chloro-1,2,3,4-tetrahydro-naphthalen-1-yl)-4,5-dihydro-1H-imidazole;
rac-2-(5-chloro-1,2,3,4-tetrahydro-naphthalen-1-yl)-4,5-dihydro-1H-imidazole;
rac-2-(7-methoxy-1,2,3,4-tetrahydro-naphthalen-1-yl)-4,5-dihydro-1H-imidazole;
rac-2-(6-methoxy-1,2,3,4-tetrahydro-naphthalen-1-yl)-4,5-dihydro-1H-imidazole;
rac-2-(5-methoxy-1,2,3,4-tetrahydro-naphthalen-1-yl)-4,5-dihydro-1H-imidazole;
rac-5-(4,5-dihydro-1H-imidazol-2-yl)-5,6,7,8-tetrahydro-naphthalen-2-ol;
rac-4-(1,2,3,4-tetrahydro-naphthalen-1-yl)-1H-imidazole;
rac-5-(4,5-dihydro-1H-imidazol-2-yl)-5,6,7,8-tetrahydro-naphthalene-2,3-diol; and
rac-5-(4,5-dihydro-1H-imidazol-2-yl)-5,6,7,8-tetrahydro-naphthalene-1,2-diol;
and a pharmaceutically acceptable carrier.

5. The pharmaceutical composition of claim 4, wherein X is N.

6. The pharmaceutical composition of claim 5, wherein Q is CH₂ and R¹ is halogen.

7. The pharmaceutical composition of claim 23, wherein the compound of formula I is selected from the group consisting of
rac-2-(5-bromo-1,2,3,4-tetrahydro-naphthalen-1-yl)-4,5-dihydro-1H-imidazole and
rac-2-(7-chloro-5-fluoro-1,2,3,4-tetrahydro-naphthalen-1-yl)-4,5-dihydro-1H-imidazole.

8. The pharmaceutical composition of claim 5, wherein Q is CH₂ and R¹ is tritium.

9. The pharmaceutical composition of claim 25, wherein the compound of formula I is rac-2-(7-tritio-1,2,3,4-tetrahydro-naphthalen-1-yl)-4,5-dihydro-1H-imidazole.

10. The pharmaceutical composition of claim 5, wherein Q is —O—.

11. The pharmaceutical composition of claim 27, wherein the compound of formula I is selected from the group consisting of
rac-2-chroman-4-yl-4,5-dihydro-1H-imidazole,
rac-2-chroman-4-yl-1H-imidazole and
rac-2-(6-fluoro-chroman-4-yl)-1H-imidazole.

12. The pharmaceutical composition of claim 5, wherein Q is O or NH and R¹ is hydrogen or halogen.

13. The pharmaceutical composition of claim 29, wherein the compound of formula I is selected from the group consisting of
rac-2-(6,8-dichloro-chroman-4-yl)-1H-imidazole and
rac-4-(1H-Imidazol-2-yl)-1,2,3,4-tetrahydro-quinoline.

14. The pharmaceutical composition of claim 4, wherein X is CH.

15. The pharmaceutical composition of claim 14, wherein Q is CH₂ and R¹ is hydrogen.

16. The pharmaceutical composition of claim 32, wherein the compound of formula I is (4-(3,4-dihydro-naphthalen-1-yl)-1H-imidazole.

17. The pharmaceutical composition of claim 14, wherein Q is O and R¹ is hydrogen.

18. The pharmaceutical composition of claim 34, wherein the compound of formula I is rac-5-chroman-4-yl-1H-imidazole hydrochloride or a tautomer thereof.

19. The pharmaceutical composition of claim 14, wherein Q is O and R¹ is lower alkyl.

20. The pharmaceutical composition of claim 35, wherein the compound of formula I is selected from the group consisting of
rac-5-(7-methyl-chroman-4-yl)-1H-imidazole or a tautomer thereof and
rac-5-(5-methyl-chroman-4-yl)-1H-imidazole or a tautomer thereof.

21. The pharmaceutical composition of claim 14, wherein Q is O and R¹ is halogen.

22. The pharmaceutical composition of claim 37, wherein the compound of formula I is selected from the group consisting of
rac-5-(6-Fluoro-chroman-4-yl)-1H-imidazole or a tautomer thereof
5-(8-Chloro-2H-chromen-4-yl)-1H-imidazole or a tautomer thereof
5-(6-Chloro-2H-chromen-4-yl)-1H-imidazole or a tautomer thereof
rac-5-(7-Fluoro-chroman-4-yl)-1H-imidazole or a tautomer thereof and
rac-5-(5-Fluoro-chroman-4-yl)-1H-imidazole or a tautomer thereof.

23. A pharmaceutical composition comprising about 0.01 mg to about 1000 mg of a compound of formula I 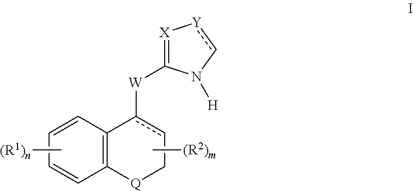
wherein
R¹ is hydrogen, tritium, hydroxy, lower alkyl, lower alkoxy, halogen, nitro, amino or lower alkyl substituted by halogen;
R² is hydrogen, hydroxy or lower alkyl;
X is N and Y is CH or CH₂ or CH-lower alkyl or
X is CH and Y is N;
Q is CH₂, O, NH, N-alkyl or N—SO₂-alkyl or N—SO₂-toluen-yl;
W is CH₂ or a bond
m and n are each independently 1, 2 or 3; when m is 2 or 3, each R² is the same or different; when n is 2 or 3, each R¹ is the same or different;
the dotted lines each independently represent an optional bond;
or a pharmaceutically active salt, racemic mixture, enantiomer, optical isomer or tautomeric form thereof, with the exception of the following compounds
rac-2-(1,2,3,4-tetrahydro-1-naphthyl)-2-imidazoline
rac-2-(7-methyl-1,2,3,4-tetrahydro-naphthalen-1-yl)-4,5-dihydro-1H-imidazole;
rac-2-(6-methyl-1,2,3,4-tetrahydro-naphthalen-1-yl)-4,5-dihydro-1H-imidazole;
rac-2-(6-chloro-1,2,3,4-tetrahydro-naphthalen-1-yl)-4,5-dihydro-1H-imidazole;
rac-2-(5-chloro-1,2,3,4-tetrahydro-naphthalen-1-yl)-4,5-dihydro-1H-imidazole;
rac-2-(7-methoxy-1,2,3,4-tetrahydro-naphthalen-1-yl)-4,5-dihydro-1H-imidazole;
rac-2-(6-methoxy-1,2,3,4-tetrahydro-naphthalen-1-yl)-4,5-dihydro-1H-imidazole;
rac-2-(5-methoxy-1,2,3,4-tetrahydro-naphthalen-1-yl)-4,5-dihydro-1H-imidazole;
rac-5-(4,5-dihydro-1H-imidazol-2-yl)-5,6,7,8-tetrahydro-naphthalen-2-ol;
rac-4-(1,2,3,4-tetrahydro-naphthalen-1-yl)-1H-imidazole;
rac-5-(4,5-dihydro-1H-imidazol-2-yl)-5,6,7,8-tetrahydro-naphthalene-2,3-diol; and
rac-5-(4,5-dihydro-1H-imidazol-2-yl)-5,6,7,8-tetrahydro-naphthalene-1,2-diol;
and a pharmaceutically acceptable carrier.

24. The pharmaceutical composition of claim 23, wherein X is N.

25. The pharmaceutical composition of claim 24, wherein Q is CH₂ and R¹ is halogen.

26. The pharmaceutical composition of claim 25, wherein the compound of formula I is selected from the group consisting of
rac-2-(5-bromo-1,2,3,4-tetrahydro-naphthalen-1-yl)-4,5-dihydro-1H-imidazole and
rac-2-(7-chloro-5-fluoro-1,2,3,4-tetrahydro-naphthalen-1-yl)-4,5-dihydro-1H-imidazole.

27. The pharmaceutical composition of claim 24, wherein Q is CH₂ and R¹ is tritium.

28. The pharmaceutical composition of claim 27, wherein the compound of formula I is rac-2-(7-tritio-1,2,3,4-tetrahydro-naphthalen-1-yl)-4,5-dihydro-1H-imidazole.

29. The pharmaceutical composition of claim 24, wherein Q is —O—.

30. The pharmaceutical composition of claim 29, wherein the compound of formula I is selected from the group consisting of
rac-2-chroman-4-yl-4,5-dihydro-1H-imidazole,
rac-2-chroman-4-yl-1H-imidazole and
rac-2-(6-fluoro-chroman-4-yl)-1H-imidazole.

31. The pharmaceutical composition of claim 24, wherein Q is O or NH and R¹ is hydrogen or halogen.

32. The pharmaceutical composition of claim 31, wherein the compound of formula I is selected from the group consisting of
rac-2-(6,8-dichloro-chroman-4-yl)-1H-imidazole and
rac-4-(1H-Imidazol-2-yl)-1,2,3,4-tetrahydro-quinoline.

33. The pharmaceutical composition of claim 23, wherein X is CH.

34. The pharmaceutical composition of claim 33, wherein Q is CH₂ and R¹ is hydrogen.

35. The pharmaceutical composition of claim 34, wherein the compound of formula I is (4-(3,4-dihydro-naphthalen-1-yl)-1H-imidazole.

36. The pharmaceutical composition of claim 33, wherein Q is O and R¹ is hydrogen.

37. The pharmaceutical composition of claim 36, wherein the compound of formula I is rac-5-chroman-4-yl-1H-imidazole hydrochloride or a tautomer thereof.

38. The pharmaceutical composition of claim 33, wherein Q is O and R¹ is lower alkyl.

39. The pharmaceutical composition of claim 38, wherein the compound of formula I is selected from the group consisting of
rac-5-(7-methyl-chroman-4-yl)-1H-imidazole or a tautomer thereof and
rac-5-(5-methyl-chroman-4-yl)-1H-imidazole or a tautomer thereof.

40. The pharmaceutical composition of claim 33, wherein Q is O and R¹ is halogen.

41. The pharmaceutical composition of claim 40, wherein the compound of formula I is selected from the group consisting of
rac-5-(6-Fluoro-chroman-4-yl)-1H-imidazole or a tautomer thereof;
5-(8-Chloro-2H-chromen-4-yl)-1H-imidazole or a tautomer thereof;
5-(6-Chloro-2H-chromen-4-yl)-1H-imidazole or a tautomer thereof;
rac-5-(7-Fluoro-chroman-4-yl)-1H-imidazole or a tautomer thereof; and
rac-5-(5-Fluoro-chroman-4-yl)-1H-imidazole or a tautomer thereof.
